(19) United States
(12) Patent Application Publication
(10) Pub. No.: US 2005/0165000 A1
(43) Pub. Date: Jul. 28, 2005

(54) Pharmaceutical compositions containing an hiv integrase inhibitor and a nonionic surfactant

(76) Inventors: Sandra K. Robertson, Harleysville, PA (US); Maria T. Cruanes, Lansdale, PA (US); Sami karaborni, Lansdale, PA (US); Drazen Ostovic, Newbury Park, CA (US); Xi-Yong Fu, Hatfield, PA (US); Ashkan Kamali, Ask West Conshohacken, PA (US); Santipharp Panmai, North Wales, PA (US)

Correspondence Address: MERCK AND CO., INC, P O BOX 2000, RAHWAY, NJ, 07065-0907, US

(21) Appl. No.: 10/509,213

(22) PCT Filed: Mar. 13, 2003
(86) PCT No.: PCT/US03/07517

Related U.S. Application Data
(60) Provisional application No. 60/371,296, filed on Apr. 10, 2002.

(51) Int. Cl. A61K 31/541; A61K 31/5377; A61K 31/496; A61K 31/4745; A61K 9/48; A61K 9/20

(52) U.S. Cl. 514/210.21; 514/233.8; 514/300; 514/227.8; 514/253.04; 424/451; 424/464

(57) ABSTRACT

Pharmaceutical compositions comprise a therapeutically effective amount of an 8-hydroxy-1,6-naphthyridine-7-carboxamide of Formula (I), or a pharmaceutically acceptable salt thereof: 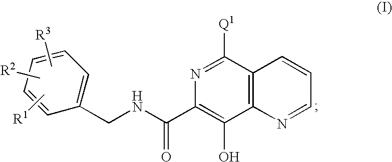
and a nonionic surfactant; wherein R¹, R², R³ and Q¹ are defined herein. Compounds of Formula (I) are HIV integrase inhibitors, and the pharmaceutical compositions are useful for preventing or treating HIV infection or for preventing, treating, or delaying the onset of AIDS. The pharmaceutical compositions are typically administered orally, for example, in the form of capsules or tablets, and can provide good oral bioavailability. Methods for preparing encapsulated and tabletted forms of the pharmaceutical compositions are described.

This application claims the benefit of U.S. Provisional Application No. 60/371,296, filed Apr. 10, 2002, the disclosure of which is hereby incorporated by reference in its entirety.

### FIELD OF THE INVENTION

The present invention is directed to pharmaceutical compositions comprising an HIV integrase inhibitor and a nonioinic surfactant. The compositions are useful for preventing or treating HIV infection and for preventing, treating or delaying the onset of AIDS. The present invention also includes methods for preparing encapsulated and tabletted forms of these pharmaceutical compositions.

### BACKGROUND OF THE INVENTION

The HIV retrovirus is the causative agent for AIDS. The HIV-1 retrovirus primarily uses the CD4 receptor (a 58 kDa transmembrane protein) to gain entry into cells, through high-affinity interactions between the viral envelope glycoprotein (gp 120) and a specific region of the CD4 molecule found in T-lymphocytes and CD4 (+) T-helper cells (Lasky L. A. et al., Cell 1987, 50: 975-985). HIV infection is characterized by an asymptomatic period immediately following infection that is devoid of clinical manifestations in the patient. Progressive HIV-induced destruction of the immune system then leads to increased susceptibility to opportunistic infections, which eventually produces a syndrome called ARC (AIDS-related complex) characterized by symptoms such as persistent generalized lymphadenopathy, fever, and weight loss, followed itself by full blown AIDS.

After entry of the retrovirus into a cell, viral RNA is converted into DNA, which is then integrated into the host cell DNA. Integration of viral DNA is an essential step in the viral life cycle. Integration is believed to be mediated by integrase, a 32 kDa enzyme, in three steps: assembly of a stable nucleoprotein complex with viral DNA sequences; cleavage of two nucleotides from the 3′ termini of the linear proviral DNA; and covalent joining of the recessed 3′ OH termini of the proviral DNA at a staggered cut made at the host target site. The fourth step in the process, repair synthesis of the resultant gap, may be accomplished by cellular enzymes.

Certain N-(benzyl)-8-hydroxy-1,6-naphthyridine-7-carboxamides are potent HIV integrase inhibitors and are useful in the prevention of infection by HIV, the treatment of infection by HIV, and in the prevention, treatment, and delay in the onset of AIDS and/or ARC, either as compounds or their pharmaceutically acceptable salts. Representative of this class of integrase inhibitors is 5-(1,1-dioxido-1,2-thiazinan-2-yl)-N-(4-fluorobenzyl)-8-hydroxy-1,6-naphthyridine-7-carboxamide (also referred to herein as “Compound A”). The structure of Compound A is as follows:

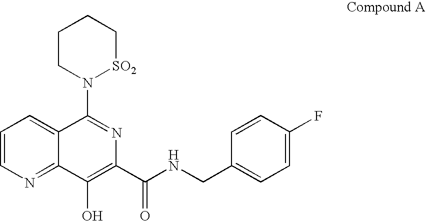

Compound A and its sodium salt (i.e., the sodium naphthyridin-8-olate) have proven difficult to formulate into orally administrable solid dosage forms (e.g., capsules and/or tablets) having a satisfactory oral bioavailability. Compound A and its sodium salt have exhibited satisfactory oral bioavailability, when administered to animals as aqueous suspensions containing a suspending agent (0.5 wt. % methocel) and an anionic surfactant (0.02 wt. % sodium lauryl sulfate). However, administration of capsules containing bulk Compound A sodium salt has resulted in substantially lower oral bioavailability than obtained with the suspension. Compound A sodium salt has also exhibited poor oral bioavailability when administered to animals in the form of wet granulated compressed tablets containing lactose (intragranular diluent), hydroxypropylcellulose (intragranular binder), Na croscarmellose (intragranular disintegrant), microcrystalline celluose (extragranular diluent), and Mg stearate (extragranular lubricant). The formulation difficulties encountered with Compound A are not unexpected in that Compound A and metal salts thereof have poor wettability and low water solubility at pH's between about 2 and about 8. Since the physiological pH typically lies within this range, the rate of the drug's dissolution in and absorption from the gastrointestinal tract can be low, particularly when the drug is administered in a solid dosage form. The same or similar formulation difficulties can be expected for other 8-hydroxy-1,6-naphthyridine-7-carboxamides of this class and their salts having low solubility at physiological pH's.

For Compound A and other integrase inhibitors of this class, there is a need for the development of pharmaceutical compositions which can be formulated into solid oral dosage forms that provide acceptable oral bioavailability.

### SUMMARY OF THE INVENTION

The present invention is directed to pharmaceutical compositions that contain an 8-hydroxy-1,6-naphthyridine-7-carboxamide HIV integrase inhibitor, are suitable for oral administration, and provide acceptable oral bioavailability. More particularly, the present invention includes a pharmaceutical composition comprising a therapeutically effective amount of a compound of Formula (I), or a pharmaceutically acceptable salt thereof: 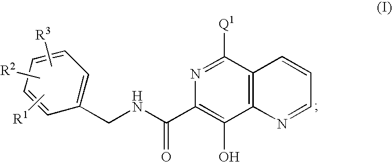
and a nonionic surfactant; wherein in Formula (I) each of R¹, R² and R³ is independently:
(1) —H,
(2) —C₁₋₆ alkyl, optionally substituted with one substituent which is —OH, —O—C₁₋₆ alkyl, —O—C₁₋₆ haloalkyl, —CN, —NO₂, —N(R^{a}R^{b}), —C(═O)N(R^{a}R^{b}), —C(═O)R^{a}, —CO₂R^{c}, —OCO₂R^{c}, —S(O)ₙR^{c}, —SO₂N(R^{a}R^{b}), —N(R^{a})C(═O)R^{b}, —N(R^{a})CO₂R^{c}, —N(R^{a})SO₂R^{c}, —N(R^{a})SO₂N(R^{a}R^{b}), —OC(═O)N(R^{a}R^{b}), or —N(R^{a})C(═O)N(R^{a}R^{b}),
(3) —O—C₁₋₆ alkyl, optionally substituted with one substituent which is —OH, —O—C₁₋₆ alkyl, —O—C₁₋₆ haloalkyl, —S(O)ₙR^{c}, —N(R^{a})—CO₂R^{c}, —C(═O)N(R^{a}R^{b}), —SO₂N(R^{a}R^{b}), —N(R^{a})C(═O)R^{b}, —N(R^{a})CO₂R^{c}, —N(R^{a})SO₂R^{c}, —N(R^{a})SO₂N(R^{a}R^{b}), —OC(═O)N(R^{a}R^{b}), or —N(R^{a})C(═O)N(R^{a}R^{b}),
(4) —C₁₋₆ haloalkyl,
(5) —O—C₁₋₆ haloalkyl,
(6) —OH,
(7) halo,
(8) —NO₂,
(9) —CN,
(10) —C(═O)R^{a},
(11) —CO₂R^{c},
(12) —S(O)ₙR^{c},
(13) —SO₂N(R^{a}R^{b}),
(14) —N(R^{a}R^{b}),
(15) —C(═O)N(R^{a}R^{b}),
(16) 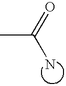
   wherein 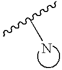
   is azetidinyl, pyrrolidinyl, piperidinyl, or morpholino,
(17) —N(R^{a})SO₂R^{c},
(18) —OC(═O)N(R^{a}R^{b}),
(19) —N(R^{a})C(═O)N(R^{a}R^{b}),
(20) —N(R^{a})—C₁₋₆ alkyl-C(═O)N(R^{a}R^{b}),
(21) —N(R^{a})—C₁₋₆ alkyl-SR^{a},
(22) —N(R^{a})—C₁₋₆ alkyl-OR^{a},
(23) —N(R^{a})—C₁₋₆ alkyl-N(R^{a})₂,
(24) —N(R^{a})—C₁₋₆ alkyl-N(R^{a})—C(R^{a})═O,
(25) —N(R^{a})—C(═O)—C₁₋₆ alkyl-N(R^{a}R^{b}),
(26) —N(R^{a})C(═O)—C(═O)N(R^{a}R^{b}),
(27) —OCO₂R^{c},
(28) —N(R^{a})—SO₂N(R^{a}R^{b}),
(29) —N(R^{a})—SO₂—C₁₋₆ alkyl-N(R^{a}R^{b}),
(30) —N(R^{a})C(═O)R^{b},
(31) —N(R^{a})CO₂R^{c},
(32) —S—C₁₋₆ alkyl-C(═O)N(R^{a}R^{b}),
(33) —N(SO₂R^{c})—C₁₋₆ alkyl-C(═O)N(R^{a}R^{b}),
(34) —N(R^{a})—C(═O)—C₁₋₆ alkyl-C(═O)N(R^{a}R^{b}),
(35) —N(R^{a})—C(═O)—C₁₋₆ alkyl-N(R^{a})C(═O)(R^{b}),
(36) —N(R^{a})—SO₂—C₁₋₆ alkyl-C(═O)N(R^{a}R^{b}),
(37) —N(R^{a})—SO₂—C₁₋₆ alkyl-N(R^{a})C(═O)(R^{b}),
(38) —C(═O)N(R^{a})—C₁₋₆ alkyl-C(═O)N(R^{a}R^{b}),
(39) —C(═O)N(R^{a})—C₁₋₆ alkyl-N(R^{a})C(═O)(R^{b}), with the proviso that the —N(Ra)— moieties are not both attached to the same carbon atom of the —C₁₋₆ alkyl- moiety,
(40) —C(═O)N(R^{a})—C₁₋₆ alkyl-O—C₁₋₃ alkyl, with the proviso that the —N(Ra)— moiety and the —O—C₁₋₃ alkyl group are not both attached to the same carbon atom of the —C₁₋₆ alkyl- moiety, or
(41) —C(═O)N(R^{a})—C₁₋₆ alkyl-S(O)ₙR^{c};
Q¹ is:
   (1) —H,
   (2) —C(═O)N(R^{a}R^{b}),
   (3) —C₁₋₆ alkyl-C(═O)N(R^{a}R^{b}),
   (4) —S—C₁₋₆ alkyl-C(═O)N(R^{a}R^{b}),
   (5) —O—C₁₋₆ alkyl-C(═O)N(R^{a}R^{b}),
   (6) —N(R^{a})—C(R^{b})═O,
   (7) —N(SO₂R^{c})—C₁₋₆ alkyl-C(═O)N(R^{a}R^{b}),
   (8) —N(R^{a})—C(═O)—C(═O)—N(R^{a}R^{b}),
   (9) —N(R^{a})SO₂R^{c},
   (10) —SO₂N(R^{a}R^{b}),
   (11) —CH═CH—C(═O)—N(R^{a}R^{b}),
   (12) —N(R^{a})—C₁₋₆ alkyl-C(═O)N(R^{a}R^{b}),
   (13) —N(R^{a})—C(═O)—N(R^{a}R^{b}),
   (14) -HetC,
   (15) —C₁₋₆ alkyl-HetC, or
   (16) —N(R^{a})—C₁₋₆ alkyl-HetC;
HetC is a 5- to 7-membered saturated heterocyclic ring containing from 1 to 4 heteratoms independently selected from N, O and S, wherein the saturated heterocyclic ring is optionally substituted with from 1 to 4 substituents each of which is independently halogen, —C₁₋₄ alkyl, —C₃₋₆ cycloalkyl, —O—C₁₋₄ alkyl, —C₁₋₄ haloalkyl, —O—C₁₋₄ haloalkyl, —CN, oxo, phenyl, benzyl, phenylethyl, —(CH₂)₀₋₃C(═O)N(R^{a}R^{b}), —(CH₂)₀₋₃C(═O)R^{a}, —N(R^{a})—C(═O)R^{b}, N(R^{a})—CO₂R^{c}, —(CH₂)₁₋₃N(R^{a})—C(═O)R^{b}, —N(R^{a}R^{b}), —(CH₂)₁₋₃N(R^{a}R^{b}), —SO₂R^{c}, —(CH₂)₀₋₃C(═O)-HetD, -HetD, —N(R^{a})-HetD, and —(CH₂)₁₋₃-HetD; wherein each HetD is independently a 5- or 6-membered heteroaromatic ring containing from 1 to 4 nitrogen atoms or a 5- or 6-membered saturated heterocyclic ring containing from 1 to 4 nitrogen atoms, wherein the ring is optionally substituted with 1 or 2 substituents each of which is independently halogen, oxo, —C₁₋₄ alkyl, or —O—C₁₋₄ alkyl;
each R^{a} is independently —H, —C₁₋₆ alkyl, —C₁₋₆ haloalkyl, or —C₃₋₆ cycloalkyl;
each R^{b} is independently —H, —C₁₋₆ alkyl, —C₁₋₆ haloalkyl, or —C₃₋₆ cycloalkyl;
each R^{c} is independently —C₁₋₆ alkyl, —C₁₋₆ haloalkyl, or —C₃₋₆cycloalkyl; and
each n is independently an integer equal to zero, 1, or 2.

The pharmaceutical compositions of the invention can be formulated into solid oral dosage forms such as capsules and tablets having good oral bioavailability. In particular, the pharmaceutical compositions of the invention can exhibit significantly enhanced oral bioavailability with respect to analogous compositions which either contain an anionic surfactant or no surfactant at all. While not wishing to be bound by a particular theory, it is believed that the nonionic surfactant improves dispersion of the drug particles (i.e., the particles of the compound of Formula I) in an aqueous medium at physiological pHs by increasing the solid surface area of the drug for dissolution mass transfer and/or to enhance solubility of the drug in the aqueous medium. The nonionic surfactant is believed to minimize drug particle flocculation and to stabilize the drug particles in a suspension state and/or increase the solubility of the drug via micellization.

The present invention also includes methods for preparing encapsulated and tabletted forms of pharmaceutical compositions of the invention. The present invention further includes use of a pharmaceutical composition of the invention for preventing or treating HIV infection or for treating or delaying the onset of AIDS.

Other embodiments, aspects and features of the present invention are either further described in or will be apparent from the ensuing description, examples and appended claims.

### DETAILED DESCRIPTION OF THE INVENTION

The pharmaceutical compositions of the present invention contain an 8-hydroxy-1,6-naphyridine-7-carboxamide of Formula (I). In one embodiment of the present invention, the pharmaceutical composition is as defined above, except that in the compound of Formula (I) (hereinafter alternatively referred to as “Compound I”), or a pharmaceutically acceptable salt thereof, each of R¹, R² and R³ is independently:
(1) —H,
(2) —C₁₋₄ alkyl, optionally substituted with one substituent which is —OH, —O—C₁₋₄ alkyl, —O—C₁₋₄ haloalkyl, —N(R^{a}R^{b}), —C(═O)N(R^{a}R^{b}), —C(═O)R^{a}, —CO₂R^{c}, —OCO₂R^{c}, —S(O)ₙR^{c}, —SO₂N(R^{a}R^{b}), —N(R^{a})C(═O)R^{b}, —N(R^{a})CO₂R^{c}, —N(R^{a})SO₂R^{c}, —N(R^{a})SO₂N(R^{a}R^{b}), —OC(═O)N(R^{a}R^{b}), or —N(R^{a})C(═O)N(R^{a}R^{b}),
(3) —O—C₁₋₄ alkyl, optionally substituted with one substituent which is —OH, —O—C₁₋₄ alkyl, —O—C₁₋₄ haloalkyl, —S(O)ₙR^{c}, —N(R^{a})—CO₂R^{c}, —C(═O)N(R^{a}R^{b}), —SO₂N(R^{a}R^{b}), —N(R^{a})C(═O)R^{b}, —N(R^{a})CO₂R^{c}, —N(R^{a})SO₂R^{c}, —N(R^{a})SO₂N(R^{a}R^{b}), —OC(═O)N(R^{a}R^{b}), or —N(R^{a})C(═O)N(R^{a}R^{b}),
(4) —C₁₋₄ haloalkyl,
(5) —O—C₁₋₄ haloalkyl,
(6) halo,
(7) —CO₂R^{c},
(8) —S(O)ₙR^{c},
(9) —SO₂N(R^{a}R^{b}),
(10) —N(R^{a}R^{b}),
(11) —C(═O)N(R^{a}R^{b}), or
(12) 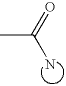
   wherein 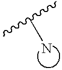
   is azetidinyl, pyrrolidinyl, piperidinyl, or morpholino;
and all other variables in Compound I are as originally defined.

In another embodiment of the present invention, the pharmaceutical composition is as originally defined above, except that in the compound of Formula (I), or a pharmaceutically acceptable salt thereof, each of R¹, R² and R³ is independently:
(1) —H,
(2) —C₁₋₄ alkyl, optionally substituted with one substituent which is —OH, —O—C₁₋₄ alkyl, —OCF₃, —N(R^{a}R^{b}), —C(═O)N(R^{a}R^{b}), —C(═O)R^{a}, —CO₂R^{c}, —OCO₂R^{c}, —S(O)ₙR^{c}, —SO₂N(R^{a}R^{b}), —N(R^{a})C(═O)R^{b}, —N(R^{a})CO₂R^{c}, —N(R^{a})SO₂R^{c}, —N(R^{a})SO₂N(R^{a}R^{b}), —OC(═O)N(R^{a}R^{b}), or —N(R^{a})C(═O)N(R^{a}R^{b}),
(3) —O—C₁₋₄ alkyl, optionally substituted with one substituent which is —OH, —O—C₁₋₄ alkyl, —OCF₃, —S(O)ₙR^{c}, —N(R^{a})—CO₂R^{c}, —C(═O)N(R^{a}R^{b}), —SO₂N(R^{a}R^{b}), —N(R^{a})C(═O)R^{b}, —N(R^{a})CO₂R^{c}, —N(R^{a})SO₂R^{c}, —N(R^{a})SO₂N(R^{a}R^{b}), —OC(═O)N(R^{a}R^{b}), or —N(R^{a})C(═O)N(R^{a}R^{b}),
(4) —CF₃,
(5) —OCF₃,
(6) halo selected from —F, —Cl, and —Br,
(7) —CO₂R^{c},
(8) —S(O)ₙR^{c},
(9) —SO₂N(R^{a}R^{b}),
(10) —N(R^{a}R^{b}), or
(11) —C(═O)N(R^{a}R^{b});
and all other variables in Compound I are as originally defined.

In still another embodiment of the present invention, the pharmaceutical composition is as originally defined above, except that in the compound of Formula (I), or a pharmaceutically acceptable salt thereof, each of R¹, R² and R³ is independently:
(1) —H,
(2) —C₁₋₄ alkyl,
(3) —(CH₂)₁₋₃—C(═O)N(R^{a}R^{b}),
(4) —(CH₂)₁₋₃—N(R^{a}R^{b}),
(5) —(CH₂)₁₋₃—CO₂R^{c},
(6) —(CH₂)₁₋₃—S(O)ₙR^{c},
(7) —(CH₂)₁₋₃—SO₂N(R^{a}R^{b}),
(8) —O—C₁₋₄ alkyl,
(9) —CF₃,
(10) —OCF₃,
(11) halo selected from —F, —Cl, and —Br,
(12) —CO₂R^{c},
(13) —S(O)ₙR^{c},
(14) —SO₂N(R^{a}R^{b}),
(15) —N(R^{a}R^{b}), or
(16) —C(═O)N(R^{a}R^{b});
with the proviso that at least one of R¹, R² and R³ is not —H;
and all other variables in Compound I are as originally defined.

In still another embodiment of the present invention, the pharmaceutical composition is as originally defined above, except that in the compound of Formula (I), or a pharmaceutically acceptable salt thereof, at least one of R¹ and R² is not —H; and R³ is —H; and all other variables in Compound I are as originally defined. In an aspect of this embodiment, R¹ is attached to the 4-position of the benzyl ring and R² is attached to the 2-position of the benzyl ring.

Another embodiment of the present invention is the pharmaceutical composition as originally defined above, except that in the compound of Formula (I), or a pharmaceutically acceptable salt thereof, Q¹ is:
(1) —H,
(2) —C(═O)N(R^{a}R^{b}),
(3) —CH₂—C(═O)N(R^{a}R^{b}),
(4) —CH₂CH₂—C(═O)N(R^{a}R^{b}),
(5) —S—CH₂—C(═O)N(R^{a}R^{b}),
(6) —O—CH₂—C(═O)N(R^{a}R^{b}),
(7) —N(R^{a})—C(R^{b})═O,
(8) —N(SO₂R^{c})—CH₂—C(═O)N(R^{a}R^{b}),
(9) —N(R^{a})—C(═O)—C(═O)—N(R^{a}R^{b}),
(10) —N(R^{a})SO₂R^{c},
(11) —SO₂N(R^{a}R^{b}),
(12) —CH═CH—C(═O)—N(R^{a}R^{b}),
(13) —N(R^{a})—CH₂—C(═O)N(R^{a}R^{b}),
(14) —N(R^{a})—C(═O)—N(R^{a}R^{b}),
(15) -HetC,
(16) —(CH₂)₁₋₃ alkyl-HetC, or
(17) —N(R^{a})—(CH₂)₁₋₃-HetC;
and all other variables in Compound I are as originally defined.

In an aspect of the preceding embodiment, HetC in Q¹ is a saturated heterocyclic ring selected from piperidinyl, morpholinyl, thiomorpholinyl, thiazolidinyl, isothiazolidinyl, oxazolidinyl, isooxazolidinyl, pyrrolidinyl, imidazolidinyl, piperazinyl, tetrahydrofuranyl, pyrazolidinyl, hexahydropyrimidinyl, thiazinanyl, thiazepanyl, thiadiazepanyl, dithiazepanyl, diazepanyl, and thiadiazinanyl, wherein the saturated heterocyclic ring is unsubstituted or substituted with 1 to 4 substituents each of which is independently:
(a) methyl or ethyl,
(b) ═O,
(c) —C(═O)N(R^{a}R^{b}),
(d) —CH₂C(═O)N(R^{a}R^{b}),
(e) —C(═O)R^{a}, or
(f) —SO₂R^{c}.

In another embodiment of the present invention, the pharmaceutical composition is as originally defined above, except that in the compound of Formula (I), or a pharmaceutically acceptable salt thereof, Q¹ is:
(1) —H,
(2) —C(═O)N(R^{a}R^{b}),
(3) —CH₂—C(═O)N(R^{a}R^{b}),
(4) —CH₂CH₂—C(═O)N(R^{a}R^{b}),
(5) —S—CH₂—C(═O)N(R^{a}R^{b}),
(6) —O—CH₂—C(═O)N(R^{a}R^{b}),
(7) —N(SO₂R^{c})—CH₂—C(═O)N(R^{a}R^{b}),
(8) —N(R^{a})—C(═O)—C(═O)—N(R^{a}R^{b}),
(9) —N(R^{a})SO₂R^{c},
(10) —CH═CH—C(═O)—N(R^{a}R^{b}),
(11) —N(R^{a})—CH₂—C(═O)N(R^{a}R^{b}),
(12) —N(R^{a})—C(═O)—N(R^{a}R^{b}),
(13) -HetC,
(14) —(CH₂)₁₋₂ alkyl-HetC, or
(15) —N(R^{a})—(CH₂)₁₋₂-HetC;
and all other variables are as originally defined.

In an aspect of the preceding embodiment, HetC in Q¹ is HetC is a saturated heterocyclic ring selected from piperidinyl, morpholinyl, thiomorpholinyl, thiazolidinyl, isothiazolidinyl, oxazolidinyl, isooxazolidinyl, pyrrolidinyl, imidazolidinyl, piperazinyl, tetrahydrofuranyl, pyrazolidinyl, hexahydropyrimidinyl, 1,2-thiazinanyl, 1,4-thiazepanyl, 1,2,5-thiadiazepanyl, 1,5,2-dithiazepanyl, 1,4-diazepanyl, and 1,2,6-thiadiazinanyl, wherein the saturated heterocyclic ring is unsubstituted or substituted with 1 to 4 substituents, each of which is independently:
(a) methyl or ethyl,
(b) ═O,
(c) —C(═O)NH₂,
(d) —C(═O)CH₃, or
(e) —SO₂CH₃.

In still another embodiment of the present invention, the pharmaceutical composition is as originally defined above, except that in the compound of Formula (I), or a pharmaceutically acceptable salt thereof, Q₁ is —H, —C(═O)N(R^{a}R^{b}), —N(R^{a})SO₂R^{c}, or 1,1-dioxido-1,2-thiazinan-2-yl; and all other variables are as originally defined.

Other embodiments of the present invention include the pharmaceutical composition as originally defined above, except that in the compound of Formula (I), or a pharmaceutically acceptable salt thereof, R¹, R² and R³ are as defined in any one of the above embodiments defining R¹, R² and R³, and Q¹ is as defined in any one of the above embodiments defining Q¹.

Other embodiments of the present invention include the pharmaceutical composition as originally defined or as defined in any of the foregoing embodiments, or an aspect thereof, wherein in the compound of Formula (I), or a pharmaceutically acceptable salt thereof,
each R^{a} is independently —H, —C₁₋₄ alkyl, or cyclopropyl;
each R^{b} is independently —H, —C₁₋₄ alkyl, or cyclopropyl; and
each R^{c} is independently a —C₁₋₄ alkyl or cyclopropyl.

Other embodiments of the present invention include the pharmaceutical composition as originally defined or as defined in any of the foregoing embodiments, or an aspect thereof, wherein Compound I or its salt is present in an amount of at least about 1 wt. % (e.g., from about 1 to about 90 wt. %, or from about 5 to about 80 wt. %) with respect to the total weight of the composition. Unless expressly stated to the contrary, any reference herein to the amount of the active ingredient (e.g., Compound I) is to the amount of the free form of the compound. Thus for example, in this embodiment, an acid salt (or base salt) of Compound I is employed in an amount which is equivalent to at least about 1 wt. % of the free base form (or free acid form, alternatively referred to herein as the “free phenol” form) of the compound.

Other embodiments of the present invention include the pharmaceutical composition as originally defined or as defined in any of the foregoing embodiments, or an aspect thereof, wherein the nonionic surfactant is present in an amount of at least about 0.1 wt. % (e.g., at least about 0.5 wt. %, or from about 0.1 to about 30 wt. %) with respect to the total weight of the composition.

Other embodiments of the present invention include the pharmaceutical composition as originally defined or as defined in any of the foregoing embodiments, or an aspect thereof, wherein the pharmaceutical composition further comprises a disintegrant.

Other embodiments of the present invention include the pharmaceutical composition as originally defined or as defined in any of the foregoing embodiments, or an aspect thereof, wherein the pharmaceutical composition further comprises a diluent.

Other embodiments of the present invention include the pharmaceutical composition as originally defined or as defined in any of the foregoing embodiments, or an aspect thereof, wherein the pharmaceutical composition further comprises a binder or a lubricant or both a binder and a lubricant.

Other embodiments of the present invention include the pharmaceutical composition as originally defined or as defined in any of the foregoing embodiments, or an aspect thereof, wherein the pharmaceutical composition further comprises an antioxidant.

The nonionic surfactant is employed to disperse the active ingredient (i.e., Compound I or a salt thereof) in order to maximize solid surface area for dissolution mass transfer. Stated another way, the nonionic surfactant is employed in the pharmaceutical compositions of the invention in an amount effective to reduce or prevent the flocculation of the active ingredient that would otherwise occur in an analogous composition containing an anionic surfactant or no surfactant at all. Suitable nonionic surfactants include polyoxyethylene castor oils, polyoxyethylene sorbitan fatty acid esters, sorbitan fatty acid esters, poloxamers, polyoxyethylene alkyl ethers, and fatty acid esters of glycerol. Exemplary polyoxyethylene castor oils include polyoxyl 35 castor oil, polyoxyl 40 castor oil, and polyoxyl 40 hydrogenated castor oil. Polyoxyethylene sorbitan fatty acid esters are partial fatty acid esters of sorbitol and its anhydrides copolymerized with various amounts of ethylene oxide. Examples of suitable polyoxyethylene sorbitan esters include polysorbate 20, polysorbate 40, polysorbate 60, and polysorbate 80. Sorbitan fatty acid esters are mixtures of partial esters of sorbitol and its mono- and/or di-anhydrides with fatty acids. Exemplary sorbitan fatty acid esters include sorbitan monoisostearate, monolaurate, monooleate, monopalmitate, monostearate, trioleate, and tristearate. Poloxamers are block copolymers of ethylene oxide and propylene oxide, examples of which include poloxamer 188, poloxamer 237, poloxamer 338, and poloxamer 407. Polyoxyalkylene alkyl ethers are polyethoxylated long chain normal alcohols (e.g., lauryl, myristyl, cetyl and stearyl alcohol). Exemplary polyoxyalkylene alkyl ethers include polyoxyl 20 cetostearyl ether, polyoxyl 10 oleyl ether, poloxy 20 oleyl ether, polyoxy 20 stearyl ether, and polyoxyl 23 lauryl ether. Exemplary glycerol fatty acid esters include glyceryl monooleate and glyceryl monostearate. In one aspect of the invention, the nonionic surfactant included in the pharmaceutical composition of the present invention is a poloxamer (e.g., poloxaminer 188 or 338 or 407) or a polysorbate (e.g., polysorbate 80). In another aspect, the nonionic surfactant is a poloxamer.

The diluent (also referred to in the art as a “filler”) is a substance used to impart bulk to the composition. A diluent can be employed, for example, to provide sufficient bulk to permit the composition to be compressed into a tablet having a practical size. Suitable diluents include anhydrous dibasic calcium phosphate, dibasic calcium phosphate dihydrate, tribasic calcium phosphate, calcium sulfate, carboxymethylcellulose calcium, microcrystalline cellulose, powdered cellulose, glucose, fructose, lactose, mannitol, dextrin, dextrose, dextrates, kaolin, lactitol, magnesium carbonate, magnesium oxide, maltitol, maltodextrin, maltose, starch, sucrose, and talc. In an aspect of the invention, the diluent employed in the pharmaceutical composition of the invention is lactose, microcrystalline cellulose, mannitol, anhydrous dibasic calcium phosphate or dibasic calcum phosphate dihyrate. In another aspect, the diluent is lactose or microcrystalline cellulose. A pharmaceutical composition of the invention (e.g., compressed tablet compositions and encapsulated granulated compositions) can contain two or more diluents (e.g., lactose and microcrystalline cellulose), which can be employed as a mixture in preparing the composition or can be added separately at the same time or can be added in separate steps in the preparation process (methods for preparing pharmaceutical compositions of the invention are described below). Accordingly, in another aspect of the present invention, the diluent comprises lactose and microcrystalline cellulose.

The disintegrant is a substance, or a mixture of substances, employed in the composition to facilitate its breakup or disintegration after administration. Suitable disintegrants include alginic acid, carboxymethylcellulose calcium, carboxymethylcellulose sodium, colloidal silicon dioxide, croscarmellose sodium, crospovidone, guar gum, magnesium aluminum silicate, methylcellulose, microcrystalline cellulose, polyacrilin potassium, povidone, sodium alginate, sodium starch glycolate, and starch. In an aspect of the invention, the disintegrant employed in the pharmaceutical composition of the invention is a superdisintegrant, such as croscarmellose sodium, crospovidone, povidone, or sodium starch glycolate. In another aspect, the disintegrant is the superdisintegrant croscarmellose sodium.

The lubricant can have one or more functions depending upon the dosage form of the composition. The lubricant can, for example, prevent adhesion of compressed tablets to the compression equipment, it can improve the flow of granules prepared via granulation of the composition prior to their compression or encapsulation, and/or it can improve the flow of an ungranulated powder in the filling of a capsule. Suitable lubricants include calcium stearate, glyceryl monostearate, glyceryl palmitostearate, hydrogenated castor oil, hydrogenated vegetable oil, light mineral oil, magnesium stearate, mineral oil, polyethylene glycol, stearic acid, talc, zinc stearate, and sodium stearyl fumarate. In an aspect of the invention, the lubricant employed in the composition of the invention is magnesium stearate or stearic acid. In another aspect, the lubricant is magnesium stearate. In still another aspect of the invention, the lubricant is magnesium stearate, stearic acid, sodium stearyl fumarate, or a combination (e.g., as a mixture in equal parts by weight) of any two of magnesium stearate, stearic acid, and sodium stearyl fumarate. In still another aspect of the invention, the lubricant is sodium stearyl fumarate. The term “combination” of lubricants refers to either the separate use of the lubricants in the same composition or to their use together as a mixture. When used separately, the lubricants in the combination can be employed concurrently or sequentially in either order, such as concurrent or sequential extragranular addition to a granulated composition.

A binder can be employed to impart cohesive qualities to the pharmaceutical composition of the invention. When the composition is formed into a compressed tablet or is granulated, a binder can be employed to ensure the tablet will remain intact after compression or that a granule will remain intact after granulation. Suitable binders include acacia, alginic acid, carboxymethylcellulose sodium, dextrin, ethylcellulose, gelatin, guar gum, hydrogenated vegetable oil (type I), hydroxyethyl cellulose, hydroxypropyl cellulose, hydroxypropyl methylcellulose, liquid glucose, magnesium aluminum silicate, maltodextrin, methylcellulose, polymethacrylates, povidone, sodium alginate, starch, and zein. In an aspect of the invention, the binder employed in the composition of the invention is hydroxypropyl cellulose, hydroxypropyl methylcellulose, or povidone. In another aspect, the binder is hydroxypropyl cellulose.

An antioxidant can be employed to prevent or minimize oxidative degradation of the active ingredient and/or other components of the pharmaceutical composition. Suitable antioxidants include a tocopherol or ester thereof, an alkyl gallate (e.g, propyl gallate), butylated hydroxyanisole (BHA), butylated hydroxytoluene (BHT), ascorbic acid, sodium ascorbate, citric acid, and sodium metabisulfite. In an aspect of the invention, the antioxidant employed in the pharmaceutical composition of the invention is BHA.

Stabilizing agents can also be employed in the pharmaceutical compositions of the invention. Stabilizing agents (also referred to as stabilizers) can be used, for example, to maintain a dispersion or minimize agglomeration of drug particles during tablet formation. Suitable stabilizing agents include carboxymethylcellulose, hydroxypropyl cellulose, hydroxypropylmethyl cellulose, and propylene glycol alginate.

Pharmaceutical compositions of the present invention can be formulated into compressed tablets or capsules. When formulating capsules or tablets, the nonionic surfactant can be brought into contact with the drug substance (Compound I or a salt thereof) as a solution (e.g., an aqueous solution). Alternatively, the drug substance and nonionic surfactant can be dry mixed before processing into a suitable dosage form.

Compressed tablets can be prepared via granulation, wherein the overall particle size of a formulation is increased through the permanent aggregation of smaller particles. Wet granulation is typically employed. Wet granulation can be accomplished, for example, by wetting a well-mixed blend of the dry ingredients (e.g., the drug substance, nonionic surfactant, diluent, disintegrant, and optionally a binder) with sufficient solvent (e.g., water or water with an alcohol co-solvent) to moisten the dry blend such that particles in the blend tack to one another to form larger particles, and then sieving, comminuting, or otherwise manipulating the size of the particles. Alternatively, wet granulation can be accomplished by employing a solution (e.g., an aqueous solution) of the nonionic surfactant to wet a well-mixed blend of the drug substance and other excipients (e.g., by spraying the surfactant-containing solution on the blend), followed by manipulating the particle size (e.g., by sieving or comminuting). However formed, the resulting wet granulate is then dried and milled into suitably sized particles (i.e., granules), which can then be compressed into tablets.

Tablets can also be formed by direct compression. For example, the nonionic surfactant can be incorporated onto the drug particles by freeze drying suspensions or solutions comprising the drug, the surfactant, and optionally (but preferably) a stabilizer; then dry blending the freeze-dried mixture with other excipients (e.g., diluent, disintegrant, and lubricant); and then directly compressing the blend into a tablet.

The compressed tablets can be sugar coated to mask any unpleasant taste or film coated to protect the tablet from atmospheric degradation. Suitable film coating suspensions include combinations of one, two or three of the following components: carboxymethylcellulose sodium, carnauba wax, cellulose acetate phthalate, cetyl alcohol, confectioner's sugar, ethyl cellulose, gelatin, hydroxyethyl cellulose, hydroxypropyl cellulose, hydroxypropyl methylcellulose, liquid glucose, maltodextrin, methyl cellulose, microcrystalline wax, Opadry I and Opadry II, polymethacrylates, polyvinyl alcohol, shellac, sucrose, talc, titanium dioxide, and zein. The films can be applied by spraying the suspension on the tablets and then drying. Film coating techniques and materials suitable for use with the present invention are described in Remington's Pharmaceutical Sciences, 18^{th} edition, edited by A. R. Gennaro, 1990, Mack Publishing Co., pp. 1665-1675.

Encapsulated pharmaceutical compositions of the present invention can be formed, for example, by freeze drying a suspension comprising the drug substance, the nonionic surfactant and optionally (but preferably) a stabilizer, then optionally blending (i.e., mixing) the freeze-dried mixture together with one or more other excipients (e.g., a disintegrant) to give a blended powder. Capsules (e.g., hard gelatin capsules) can then be filled with a suitable amount of the blended powder and sealed. Alternatively, the ingredients can be formed into granules via wet granulation as described above and the capsules filled with a suitable amount of the granules and sealed. The use of granules is preferred when the ungranulated powder has poor bulk flow properties.

Technology and equipment suitable for preparing solid dosage forms of the pharmaceutical compositions of the present invention (e.g., capsules and compressed tablets) are described in Remington's Pharmaceutical Sciences, 18^{th} edition, edited by A. R. Gennaro, 1990, Chapter 89.

The present invention includes an encapsulated pharmaceutical composition comprising a compound of Formula (I) as originally defined above or a pharmaceutically acceptable salt thereof, a nonionic surfactant, a disintegrant, optionally a diluent, optionally a binder, optionally a lubricant, and optionally an antioxidant. Embodiments of the encapsulated composition include encapsulated compositions in which any one or more of Compound I (or a pharmaceutically acceptable salt thereof) and the other ingredients is (are) independently as defined in any of the embodiments or aspects of the pharmaceutical composition of the invention set forth above. In another embodiment, the encapsulated composition is granulated, wherein the granulated composition comprises from about 5 to about 80 wt. % Compound I or a salt thereof, from about 0.1 to about 20 wt. % nonionic surfactant, from 0 to about 90 wt. % diluent, from about 0.5 to about 10 wt. % disintegrant, from 0 to about 20 wt. % (e.g., from 0 to about 10 wt. %) binder, from 0 to about 10 wt. % lubricant, and from about 0 to about 0.1 wt. % antioxidant. In an aspect of this embodiment, the granulated composition is as just defined, except that the lubricant is present in an amount of from about 0 to about 6 wt. %. In a feature of this aspect, the lubricant is present in an amount of from about 0 to about 2 wt. % In another embodiment, the encapsulated composition is granulated, wherein the granulated composition comprises from about 5 to about 80 wt. % Compound I or a salt thereof, from about 0.1 to about 20 wt. % nonionic surfactant, from 0 to about 90 wt. % diluent, from about 0.5 to about 10 wt. % disintegrant, from 0 to about 20 wt. % (e.g., from 0 to about 10 wt. %) binder, and from 0 to about 2 wt. % lubricant. In an aspect of this embodiment, the granulated composition is as just defined, except that the lubricant is present in an amount of from about 0.2 to about 2 wt. % and/or the binder is present in an amount of from about 0.1 to about 20 wt. %. In still another embodiment, the encapsulated composition is granulated, wherein the granulated composition comprises from about 5 to about 40 wt. % Compound I or a salt thereof, from about 0.5 to about 15 wt. % (e.g., from about 0.5 to about 10 wt. %) nonionic surfactant (e.g., poloxamer), from about 0.5 to about 10 wt. % disintegrant (e.g., croscarmellose sodium), from about 30 to about 90 wt. % diluent (e.g., lactose, microcrystalline cellulose, or both lactose and microcrystalline cellulose, e.g., in equal parts by weight), from 0 to about 20 wt. % (e.g., from 0 to about 10 wt. %) binder (e.g., hydroxypropyl cellulose), and from 0 to about 6 wt. % (e.g., from about 0.2 to about 6 wt. % or from 0 to about 2 wt. %) lubricant (e.g., magnesium stearate, sodium stearyl fumarate, or a combination of magnesium stearate and sodium stearyl fumarate, such as a mixture thereof in equal parts by weight). In an aspect of this embodiment, the granulated composition is as just defined, except that the lubricant is present in an amount of from about 0.5 to about 2 wt. % and/or the binder is present in an amount of from about 0.2 to about 20 wt. %. In yet another embodiment, the encapsulated composition is granulated, wherein the granulated composition is as defined in the preceding embodiment, except that the diluent is employed in an amount of from about 20 to about 80 wt. % (e.g., lactose, microcrystalline cellulose, or both lactose and microcrystalline cellulose, such as from about 10 to about 40 wt. % lactose and from about 10 to about 40 wt. % microcrystalline cellulose).

Unless otherwise indicated, weight percents herein are based on the total weight of all the components in the composition.

The present invention includes a compressed tablet pharmaceutical composition comprising a compound of Formula (I) as originally defined above or a pharmaceutically acceptable salt thereof, a nonionic surfactant, diluent, a disintegrant, a lubricant, optionally a binder, and optionally an antioxidant. Embodiments of the compressed tablet composition include compressed tablet compositions in which any one or more of Compound I (or a pharmaceutically acceptable salt thereof) and the other ingredients is (are) independently as defined in any of the embodiments or aspects of the pharmaceutical composition of the invention set forth above. In another embodiment, the compressed tablet comprises from about 5 to about 75 wt. % compound of Formula (I), from about 0.1 to about 20 wt. % nonionic surfactant, from about 15 to about 90 wt. % diluent, from about 0.5 to about 10 wt. % disintegrant, from about 0.2 to about 10 wt. % lubricant, from 0 to about 10 wt. % binder, and from 0 to about 0.1 wt. % antioxidant. In an aspect of this embodiment, the compressed tablet composition is as just defined, except that the lubricant is present in an amount of from about 0.2 to about 6 wt. %. In a feature of this aspect, the lubricant is employed in an amount of from about 0.2 to about 2 wt. %. In another aspect of this embodiment, the compressed tablet composition is as originally defined in the embodiment, except that the binder is present in an amount of from about 0.5 to about 5 wt. % and/or the antioxidant is present in an amount of from about 0.01 to about 0.1 wt. %. In still another embodiment, the compressed tablet composition comprises from about 10 to about 70 wt. % (e.g., from about 10 to about 50 wt. %) Compound I or a pharmaceutically acceptable salt thereof, from about 0.5 to about 10 wt. % nonionic surfactant (e.g., poloxamer), from about 10 to about 50 wt. % of a first diluent (e.g., lactose), from about 10 to about 50 wt. % of a second diluent (e.g., microcrystalline cellulose), from about 0.5 to about 10 wt. % disintegrant (e.g., croscarmellose sodium), from about 0.2 to about 6 wt. % lubricant (e.g., magnesium stearate, sodium stearyl fumarate, or a combination of magnesium stearate and sodium stearyl fumarate such as a mixture thereof in equal parts by weight), from 0 to about 5 wt. % binder (e.g., hydroxypropyl cellulose), and from 0 to about 0.1 wt. % antioxidant (e.g., BHA). In still another embodiment, the compressed tablet composition comprises from about 10 to about 30 wt. % Compound I or a pharmaceutically acceptable salt thereof, from about 0.5 to about 10 wt. % nonionic surfactant (e.g., poloxamer), from about 15 to about 50 wt. % of a first diluent (e.g., lactose), from about 15 to about 50 wt. % of a second diluent (e.g., microcrystalline cellulose), from about 0.5 to about 5 wt. % disintegrant (e.g., croscarmellose sodium), from about 0.2 to about 2 wt. % lubricant (e.g., magnesium stearate), from 0 to about 5 wt. % binder (e.g., hydroxypropyl cellulose), and from 0 to about 0.1 wt. % antioxidant (e.g., BHA). In an aspect of this embodiment, the compressed tablet composition is as just defined, except that the binder is present in an amount of from about 0.5 to about 5 wt. % and/or the antioxidant is present in an amount of from about 0.01 to about 0.1 wt. %.

A first class of the present invention includes any pharmaceutical composition comprising a therapeutically effective amount of a compound of Formula (II) (hereinafter alternatively referred to as “Compound II”) or a pharmaceutically acceptable salt thereof: 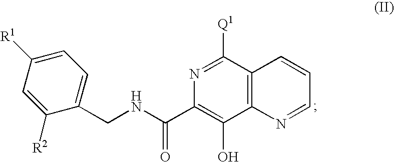
and a nonionic surfactant; wherein in Formula (II) each of R¹ and R² is independently:
(1) —H,
(2) —C₁₋₄ alkyl,
(3) —(CH₂)₁₋₃—C(═O)N(R^{a}R^{b}),
(4) —(CH₂)₁₋₃—N(R^{a}R^{b}),
(5) —(CH₂)₁₋₃—CO₂R^{c},
(6) —(CH₂)₁₋₃—S(O)ₙR^{c},
(7) —(CH₂)_{1 3}—SO₂N(R^{a}R^{b}),
(8) —O—C₁₋₄ alkyl,
(9) —CF₃,
(10) —OCF₃,
(11) halo selected from —F, —Cl, and —Br,
(12) —CO₂R^{c},
(13) —S(O)ₙR^{c},
(14) —SO₂N(R^{a}R^{b}),
(15) —N(R^{a}R^{b}), or
(16) —C(═O)N(R^{a}R^{b});
with the proviso that at least one of R¹ and R² is not —H;
Q¹ is —H, —C(═O)N(R^{a}R^{b}), —N(R^{a})SO₂R^{c}, or 1,1-dioxido-1,2-thiazinan-2-yl;
each R^{a} is independently —H, —C₁₋₄ alkyl, or cyclopropyl;
each R^{b} is independently —H, —C₁₋₄ alkyl, or cyclopropyl; and
each R^{c} is independently a —C₁₋₄ alkyl or cyclopropyl.

In an aspect of the first class, the pharmaceutical composition is as defined in the class, except that in the compound of Formula (II), or a pharmaceutically acceptable salt thereof, each of R¹ and R² is independently:
(1) —H,
(2) —C₁₋₄ alkyl,
(3) —(CH₂)₁₋₃—C(═O)N(R^{a}R^{b}),
(4) —(CH₂)₁₋₃—CO₂R^{c},
(5) —(CH₂)₁₋₃—SO₂R^{c},
(6) —(CH₂)₁₋₃—SR^{c},
(7) —(CH₂)₁₋₃—SO₂N(R^{a}R^{b}),
(8) —O—C₁₋₄ alkyl,
(9) —CF₃,
(10) —OCF₃,
(11) halo selected from —F, —Cl, and —Br,
(12) —CO₂R^{c},
(13) —SO₂R^{c},
(14) —SO₂N(R^{a}R^{b}), or
(15) —C(═O)N(R^{a}R^{b});
with the proviso that at least one of R¹ and R² is not —H;
Q¹ is 1,1-dioxido-1,2-thiazinan-2-yl;
each R^{a} is independently —H or —C₁₋₄ alkyl;
each R^{b} is independently —H or —C₁₋₄ alkyl; and
each R^{c} is independently —C₁₋₄ alkyl.

In another aspect of the first class, the pharmaceutical composition is as originally defined in the class, except that the compound of Formula (II) is a compound selected from the group consisting of:
Compound A, which is 5-(1,1-dioxido-1,2-thiazinan-2-yl)-N-(4-fluorobenzyl)-8-hydroxy-1,6-naphthyridine-7-carboxamide;
Compound B, which is N-{4-fluoro-2-[(methylamino)carbonyl]benzyl}-5-(1,1-dioxido-1,2-thiazinan-2-yl)-8-hydroxy-1,6-naphthyridine-7-carboxamide;
and pharmaceutically acceptable salts thereof.

In still another aspect of the first class, the pharmaceutical composition is as originally defined in the first class, except that Compound II is Compound A, a sodium salt of Compound A, Compound B, or a sodium salt of Compound B.

In still another aspect of the first class, the pharmaceutical composition is as originally defined in the first class, except that Compound II is a sodium salt of Compound A.

In still another aspect of the first class, the pharmaceutical composition is as originally defined in the first class, except that Compound II is a sodium salt of Compound A having a mean particle size of from about 1 to about 20 μm (e.g., from about 1 to about 10 μm).

In still another aspect of the first class, the pharmaceutical composition is as originally defined in the first class, except that Compound II is a potassium salt of Compound B. In a feature of this aspect, Compound II is a potassium ethanolate salt of Compound B. In another feature of this aspect, Compound II is a potassium ethanolate hydrate salt of Compound B. In sub-features of each of the foregoing features, the salt is crystalline, e.g., prepared by treating an ethanol solution of Compound B with KOH (e.g., aqueous KOH) and then crystallizing the ethanolate or ethanolate hydrate.

Other aspects of the first class of the present invention include the pharmaceutical composition as originally defined in the first class or as defined in any one of the foregoing aspects thereof, incorporating one or more of the following features (a) to (i):
(a1) the nonionic surfactant comprises a poloxamer or a polysorbate;
(a2) the nonionic surfactant comprises a poloxamer;
(a3) the nonionic surfactant is present in an amount of at least about at least about 0.1 wt. % (e.g., at least about 0.5 wt. %, or from about 0.1 to about 30 wt. %);
(a4) the nonionic surfactant is present in an amount of at least about at least about 1 wt. % (e.g., from about 1 to about 30 wt. %);
(a5) the nonionic surfactant comprises a poloxamer or a polysorbate and is present in an amount set forth in (a3) or (a4);
(b1) the pharmaceutical composition further comprises a disintegrant;
(b2) the pharmaceutical composition further comprises a disintegrant which comprises croscarmellose sodium, crospovidone, povidone, or sodium starch glycolate;
(b3) the pharmaceutical composition further comprises a disintegrant which comprises croscarmellose sodium;
(c1) the pharmaceutical composition further comprises a diluent;
(c2) the pharmaceutical composition further comprises a diluent which comprises lactose, microcrystalline cellulose, mannitol, anhydrous dibasic calcium phosphate, dibasic calcum phosphate dihyrate, or a combination of any two of the foregoing;
(c3) the pharmaceutical composition further comprises a diluent which comprises lactose, microcrystalline cellulose, or both lactose and microcrystalline cellulose;
(d1) the pharmaceutical composition further comprises a binder;
(d2) the pharmaceutical composition further comprises a binder which comprises hydroxypropyl cellulose, hydroxypropyl methylcellulose, or povidone;
(d3) the pharmaceutical composition further comprises a binder which comprises hydroxypropyl cellulose;
(e1) the pharmaceutical composition further comprises a lubricant;
(e2) the pharmaceutical composition further comprises a lubricant which comprises magnesium stearate, stearic acid, sodium stearyl fumarate, or a combination (e.g., as a mixture in equal parts by weight) of any two of magnesium stearate, stearic acid and sodium stearyl fumarate;
(e3) the pharmaceutical composition further comprises a lubricant which comprises sodium stearyl fumarate;
(f1) the pharmaceutical composition further comprises an antioxidant;
(f2) the pharmaceutical composition further comprises an antioxidant which comprises BHA;
(g1) Compound II (or Compound A or B) or its salt is present in the composition in an amount of from about 5 to about 500 mg; or
(g2) Compound II (or Compound A or B) or its salt is present in the composition in an amount of from about 10 to about 100 mg;
(g3) Compound II (or Compound A or B) or its salt is present in the composition in an amount of from about 10 to about 200 mg;
(g4) Compound II (or Compound A or B) or its salt is present in the composition in an amount of from about 5 to about 400 mg; or
(h1) the pharmaceutical composition is encapsulated;
(h2) the pharmaceutical composition is encapsulated, wherein the encapsulated composition is a granulated composition;
(h3) the pharmaceutical composition is encapsulated, wherein the encapsulated composition is a granulated composition in which Compound II (or A or B) or its salt is present in an amount of from about 1 to about 90 wt. % (e.g., from about 5 to about 80 wt. %);
(h4) the pharmaceutical composition is encapsulated, wherein the encapsulated composition is granulated using a wet granulation step;
(h5) the pharmaceutical composition is encapsulated, wherein the encapsulated composition is a granulated composition further comprising a disintegrant, optionally a diluent (e.g., a single diluent or a combination of two diluents), optionally a binder, optionally a lubricant (e.g., a single lubricant or a combination of two lubricants), and optionally an antioxidant (e.g., no antioxidant);
(h6) the encapsulated pharmaceutical composition is as set forth in (h5) and includes the lubricant, wherein the encapsulated composition is granulated using a wet granulation step, with extragranular addition of the lubricant;
(h7) the pharmaceutical composition is encapsulated, wherein the encapsulated composition is a granulated composition which comprises from about 5 to about 80 wt. % Compound II (or A or B) or a pharmaceutically acceptable salt thereof, from about 0.1 to about 20 wt. % nonionic surfactant, from 0 to about 90 wt. % diluent, from about 0.5 to about 10 wt. % disintegrant, from 0 to about 20 wt. % (e.g, from about 0.2 to about 20 wt. %) binder, and from 0 to about 10 wt. % (e.g., from 0 to about 6 wt. %, from 0 to about 2 wt. %, or from about 0.2 to about 2 wt. %) lubricant; and from 0 to about 0.1 wt. % antioxidant (e.g., no antioxidant);
(h8) the pharmaceutical composition is encapsulated, wherein the encapsulated composition is a granulated composition further comprising a disintegrant, optionally a diluent, optionally a binder, and optionally a lubricant; and wherein the composition is granulated by
   (A) wet granulating a mixture of Compound II (or A or B) or its pharmaceutically acceptable salt, the nonionic surfactant, the disintegrant, the diluent (optional), and the binder (optional); and optionally then milling the wet granulated mixture;
   (B) drying the wet granulated mixture of Step A;
   (C) milling the dried mixture of Step B; and
   (D) optionally lubricating the milled mixture of Step C with the lubricant;
(h9) the pharmaceutical composition is an encapsulated and granulated composition as set forth in (h7) and is granulated as set forth in (h8);
(i1) the pharmaceutical composition is compressed into a tablet;
(i2) the pharmaceutical composition is compressed into a tablet in which Compound II (or A or B) or its salt is present in an amount of from about 1 to about 80 wt. % (e.g., from about 5 to about 75 wt. %);
(i3) the pharmaceutical composition is compressed into a tablet, wherein the compressed tablet further comprises a diluent (e.g., a single diluent or a combination of two diluents), a disintegrant, a lubricant (e.g., a single lubricant or a combination of two lubricants), optionally a binder, and optionally an antioxidant;
(i4) the pharmaceutical composition is compressed into a tablet which comprises from about 5 to about 75 wt. % Compound II (or A or B) or its pharmaceutically acceptable salt, from about 0.1 to about 20 wt. % nonionic surfactant, from about 15 to about 90 wt. % diluent, from about 0.5 to about 10 wt. % (e.g., from about 0.5 to 5 wt. %) disintegrant, from about 0.2 to about 10 wt. % (e.g., from about 0.2 to about 6 wt. %, or from about 0.2 to about 2 wt. %) lubricant, from 0 to about 10 wt. % (e.g., from 0 to about 5 wt. %, or from about 0.5 to about 5 wt. %) binder, and from 0 to about 0.1 wt. % (e.g., from about 0.01 to about 0.1 wt. %) antioxidant;
(i5) the pharmaceutical composition is compressed into a tablet, wherein the compressed tablet further comprises a diluent (or a first and a second diluent such as lactose and microcrystalline cellulose), a disintegrant, a lubricant, optionally a binder, and optionally an antioxidant; and wherein the compressed tablet is prepared by:
   (A) wet granulating a mixture of Compound II (or A or B) or its pharmaceutically acceptable salt, the nonionic surfactant, the diluent (or the first diluent and the second diluent), the disintegrant, the binder (optional), and the antioxidant (optional); and optionally then milling the wet granulated mixture;
   (B) drying the wet granulated mixture of Step A;
   (C) milling the dried mixture of Step B;
   (D) lubricating the milled mixture of Step C with the lubricant; and
   (E) compressing the lubricated mixture of Step D into a tablet.
(i6) the pharmaceutical composition is compressed into a tablet which comprises the composition as set forth in (i4) and is prepared as set forth in (i5);
(i7) the pharmaceutical composition is compressed into a tablet, wherein the compressed tablet further comprises a first diluent (or diluent A), a second diluent (or diluent B), a disintegrant, a lubricant, optionally a binder, and optionally an antioxidant; and wherein the compressed tablet is prepared by:
   (A) wet granulating a mixture of Compound II or its pharmaceutically acceptable salt, the nonionic surfactant, the first diluent, the disintegrant, the binder (optional), and the antioxidant (optional); and optionally then milling the wet granulated mixture;
   (B) drying the wet granulated mixture of Step A;
   (C) milling the dried mixture of Step B;
   (D) blending the milled mixture of Step C with the second diluent;
   (E) lubricating the blended mixture of Step D with the lubricant; and
   (F) compressing the lubricated mixture of Step E into a tablet;
(i8) the pharmaceutical composition is compressed into a tablet which comprises from about 10 to about 50 wt. % (e.g., from about 10 to about 30 wt. %) Compound II or its pharmaceutically acceptable salt, from about 0.5 to about 10 wt. % nonionic surfactant, from about 10 to about 50 wt. % (e.g., from about 15 to about 50 wt. %) of a first diluent, from about 10 to about 50 wt. % (e.g., from about 15 to about 50 wt. %) of a second diluent, from about 0.5 to about 10 wt. % (e.g., from about 0.5 to about 5 wt. %) disintegrant, from about 0.2 to about 6 wt. % (e.g., from about 0.2 to about 2 wt. %) lubricant, from 0 to about 5 wt. % binder (e.g., from about 0.5 to 5 wt. %), and from 0 to about 0.1 wt. % (e.g, from about 0.01 to about 0.1 wt. %) antioxidant;
(i9) the pharmaceutical composition is compressed into a tablet which comprises the composition as set forth in (i8) and is prepared as set forth in (i7); and
(i10) the pharmaceutical composition is compressed into a tablet as set forth in any of (i1) to (i9), and the compressed tablet is film coated.

It is of course understood that certain of the preceding features are mutually exclusive and cannot be incorporated into the same pharmaceutical composition. For example, the pharmaceutical composition can either be encapsulated (feature h1) or compressed into a tablet (feature i1), but cannot simultaneously be formed into both a capsule and a tablet. Such incompatible combinations of features can be readily identified by the person of ordinary skill in the art and are not included among the aspects of the first class.

A second class of the present invention includes any encapsulated pharmaceutical composition comprising a therapeutically effective amount of Compound A or a sodium salt thereof, and nonionic surfactant; wherein Compound A is:

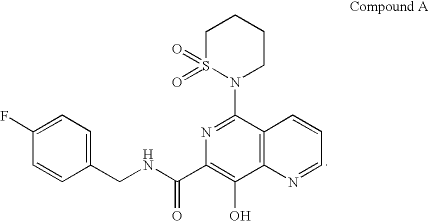

In an aspect of the second class, the encapsulated pharmaceutical composition comprises a therapeutically effective amount of a sodium salt of Compound A and nonionic surfactant.

In another aspect of the second class, the encapsulated pharmaceutical composition comprises a therapeutically effective amount of a sodium salt of Compound A and nonionic surfactant, wherein the sodium salt of Compound A has a mean particle size of from about 1 to about 10 microns.

Other aspects of the second class of the present invention include the encapsulated pharmaceutical composition as originally defined in the second class or as defined in any one of the foregoing aspects thereof, incorporating any one or more features corresponding to features (a) to (h) of the first class as set forth above.

A third class of the present invention includes any compressed tablet pharmaceutical composition comprising a therapeutically effective amount of Compound A or a sodium salt thereof, and a nonionic surfactant.

In an aspect of the third class, the compressed tablet composition comprises a therapeutically effective amount of a sodium salt of Compound A and nonionic surfactant.

In another aspect of the third class, the compressed tablet composition comprises a therapeutically effective amount of a sodium salt of Compound A and nonionic surfactant, wherein the sodium salt of Compound A has a mean particle size of from about 1 to about 10 microns.

Other aspects of the third class of the present invention include the compressed tablet pharmaceutical composition as originally defined in the third class or as defined in any one of the foregoing aspects thereof, incorporating any one or more features corresponding to features (a) to (g) and (i) of the first class as set forth above.

The present invention also includes a method for preparing a compressed tablet pharmaceutical composition comprising a therapeutically effective amount of Compound I or a pharmaceutically acceptable salt thereof, nonionic surfactant, diluent A, disintegrant, and lubricant; wherein the method comprises:
(A) wet granulating a mixture of Compound I or its salt, the nonionic surfactant, diluent A, and the disintegrant; and optionally then milling the wet granulated mixture;
(B) drying the wet granulated mixture of Step A;
(C) milling the dried mixture of Step B; and
(D) lubricating the milled mixture of Step C with the lubricant; and
(E) compressing the lubricated mixture of Step D into a tablet.

Embodiments of this process include the process as just described incorporating one or more of the features (a*) to (g*) as follows:
(a1*) the compound is Compound II or a pharmaceutically acceptable salt thereof;
(a2*) the compound is selected from the group consisting of Compound A, Compound B, and pharmaceutically acceptable salts thereof (e.g., the sodium salts);
(a3*) the compound is Compound A sodium salt;
(a4*) the compound is a crystalline sodium salt of Compound A;
(a5*) the compound is Compound I (or Compound II or Compound A or Compound B) having a mean particle size of from about 1 to about 20 microns (e.g., the crystalline sodium salt of Compound A having a mean particle size of from about 1 to about 10 microns);
(b1*) the nonionic surfactant is a poloxamer or a polysorbate;
(b2*) the nonionic surfactant is a poloxamer (e.g., poloxamer 338 or poloxamer 407);
(c1*) diluent A is lactose, microcrystalline cellulose, mannitol, anhydrous dibasic calcium phosphate, dibasic calcum phosphate dihyrate, or a combination of any two of the foregoing;
(c2*) diluent A is lactose, microcrystalline cellulose, or both lactose and microcrystalline cellulose;
(d1*) the disintegrant is croscarmellose sodium, crospovidone, povidone, or sodium starch glycolate;
(d2*) the disintegrant is croscarmellose sodium;
(e*) the lubricant is magnesium stearate, sodium stearyl fumarate, or a combination of magnesium stearate and sodium stearyl fumarate;
(f1*) the compressed tablet composition further comprises diluent B (e.g., microcrystalline cellulose), optionally a binder (e.g., hydroxypropyl cellulose), and optionally an antioxidant (e.g, BHA); and wherein the mixture employed in granulation Step A comprises Compound I (or II or A or B) or its salt, the nonionic surfactant, the disintegrant, diluent A, diluent B, the binder (optional), and the antioxidant (optional);
(f2*) the compressed tablet composition further comprises diluent B (e.g., microcrystalline cellulose), optionally a binder (e.g., hydroxypropyl cellulose), and optionally an antioxidant (e.g., BHA); wherein the mixture employed in granulation Step A comprises Compound I (or II or A or B) or its salt, the nonionic surfactant, the disintegrant, diluent A, the binder (optional), and the antioxidant (optional); and wherein the method further comprises blending the milled mixture of Step C with diluent B prior to lubrication in Step D;
(f3*) the compressed tablet composition is as set forth in (f1*) or (f2*), wherein in Step A the mixture that is wet granulated is prepared by mixing an aqueous solution of the nonionic surfactant and the antioxidant (optional), with a dry blend of Compound I (or II or A or B) or its salt, diluent A and diluent B in (f1*) or diluent A in (f2*), distintegrant, and the binder (optional);

(f4*) the compressed tablet composition is as set forth in (f1*) or (f2*), wherein in Step A the mixture that is wet granulated is prepared by mixing a granulating fluid comprising water, alcohol (e.g., C₁₋₄ alkyl alcohols such as ethanol), or an alcohol-water mixture (e.g., 5 to 95 wt. % ethanol-95 to 5 wt. % water solution, such as 95 wt. % ethanol-5 wt. % water) and also containing the optional antioxidant, with a dry blend of Compound I (or II or A or B) or its salt, nonionic surfactant, diluent A as originally defined or diluent A and diluent B in (f1*) or diluent A in (f2*), disintegrant, and the binder (optional);
(f5*) the compressed tablet resulting from Step E is the composition set forth in (f1*), (f2*), (f3*), or (f4*) and comprises from about 10 to about 70 wt. % (e.g., from about 10 to about 50 wt. % or from about 10 to about 30 wt. %) Compound I (or II or A or B) or its pharmaceutically acceptable salt, from about 0.5 to about 10 wt. % nonionic surfactant (e.g., poloxamer such as poloxamer 407), from about 10 to about 50 wt. % (e.g., from about 15 to about 50 wt. %) diluent A (e.g., lactose), from about 10 to about 50 wt. % (e.g., from about 15 to about 50 wt. %) diluent B (e.g., microcrystalline cellulose), from about 0.5 to about 10 wt. % (e.g., from about 0.5 to about 5 wt. %) disintegrant (e.g., croscarmellose sodium), from about 0.2 to about 6 wt. % (e.g., from about 0.2 to about 2 wt. %) lubricant (e.g., magnesium stearate or sodium stearyl fumarate or both), from 0 to about 5 wt. % (e.g., from about 0.5 to about 10 wt. %, from about 0 to about 5 wt. %, or from about 0.5 to about 5 wt. %) binder (e.g., hydroxypropyl cellulose), and from 0 to about 0.1 wt. % (e.g, from about 0.01 to about 0.1 wt. %) antioxidant (e.g., BHA);
(g1*) the method further comprises: (F) coating the compressed tablet; and
(g2*) the method further comprises: (F) coating the compressed tablet with a film coating suspension to afford a coated tablet in which the coating is from about 1 to about 5% of the weight of the compressed tablet.

The present invention also includes a compressed tablet pharmaceutical composition prepared by the method comprising Steps A to E (and optionally F) as just described.

The present invention also includes a method for preparing a compressed tablet pharmaceutical composition comprising a therapeutically effective amount of Compound I or a pharmaceutically acceptable salt thereof, nonionic surfactant, diluent, disintegrant, lubricant, and a stabilizing agent; wherein the method comprises:
(A) freeze-drying a suspension (e.g., an aqueous suspension) of Compound I or its salt, the nonionic surfactant, and the stabilizing agent;
(B) dry mixing the freeze-dried product of Step A with the diluent, disintegrant, and lubricant; and
(C) compressing the mixture of Step B into a tablet.

The freeze-drying of the suspension can be accomplished using conventional techniques such as those described in Remington's Pharmaceutical Sciences, 18^{th} edition, edited by A. R. Gennaro, 1990, Mack Publishing Co., pp. 1565-1567. Embodiments of this process include the process as just described incorporating one or more features corresponding to features (a*) to (e*) and (g*) set forth above and/or incorporating one or more of the following features:
(h*) the stabilizing agent employed in Step A is hydroxypropylcellulose;
(i1*) the compressed tablet resulting from Step C comprises from about 5 to about 50 wt. % Compound I (or Compound II or Compound A or Compound B) or its salt, from about 0.5 to about 10 wt. % nonionic surfactant (e.g., poloxamer), from about 15 to about 90 wt. % diluent (e.g., lactose), from about 0.5 to about 5 wt. % disintegrant (e.g., croscarmellose sodium), from about 0.2 to about 2 wt. % lubricant (e.g., magnesium stearate), and from about 0.5 to about 5 wt. % stabilizing agent (e.g., hydroxypropyl cellulose);
(i2*) the compressed tablet resulting from Step C comprises from about 5 to about 75 wt. % Compound I (or II or A or B) or its salt, from about 0.1 to about 20 wt. % nonionic surfactant, from about 15 to about 90 wt. % diluent, from about 0.5 to about 10 wt. % disintegrant, from about 0.2 to about 6 wt. % lubricant, and from about 0.5 to about 5 wt. % stabilizing agent;
(j*) the compressed tablet further comprises an antioxidant (e.g., BHA), wherein the antioxidant is included in the suspension that is freeze-dried in Step A; and
(k*) in Step B, the freeze-dried product of Step A is first mixed with the diluent and disintegrant, followed by addition of and mixing with the lubricant.

The present invention also includes a compressed tablet pharmaceutical composition prepared by the method comprising Steps A to C as just described.

The present invention includes still another method for preparing a compressed tablet that combines the freeze-drying approach just described with the wet granulation previously described. More particularly, the present invention includes a method for preparing a compressed tablet pharmaceutical composition comprising a therapeutically effective amount of Compound I or a pharmaceutically acceptable salt thereof, nonionic surfactant, diluent A, diluent B, disintegrant, lubricant, a stabilizing agent, and a binder; wherein the method comprises:
(A) freeze-drying an aqueous suspension of Compound I or its salt, the nonionic surfactant, and the stabilizing agent;
(B) dry mixing the freeze-dried product of Step A with diluent A, disintegrant, and binder;
(C) wet granulating the mixture of Step B;
(D) drying the wet granulated mixture of Step C;
(E) milling the dried mixture of Step D;
(F) blending the milled mixture of Step E with diluent B;
(G) lubricating the blended mixture of Step F with the lubricant; and
(H) compressing the lubricated mixture of Step G into a tablet.

Embodiments of this process include the process as just described incorporating one or more features corresponding to features (a*) to (e*), (g*), and (h*) as set forth above.

The present invention also includes a method for preparing an encapsulated pharmaceutical composition comprising a therapeutically effective amount of Compound I or a pharmaceutically acceptable salt thereof, nonionic surfactant and disintegrant; wherein the method comprises:
(A) wet granulating a mixture of Compound I or its salt, the nonionic surfactant, and the disintegrant; and optionally then milling the wet granulated mixture;
(B) drying the wet granulated mixture of Step A;
(C) milling the dried mixture of Step B; and
(D) encapsulating the milled mixture of Step C.

Embodiments of this process include the process as just described incorporating one or more of the features (aa) to (ee) as follows:
(aa1) the compound is Compound II or a pharmaceutically acceptable salt thereof;
(aa2) the compound is selected from the group consisting of Compound A, Compound B, and pharmaceutically acceptable salts thereof (e.g., the sodium salts);
(aa3) the compound is Compound A sodium salt;
(aa4) the compound is a crystalline sodium salt of Compound A;
(aa5) the compound is Compound I (or Compound II or Compound A or Compound B) having a mean particle size of from about 1 to about 20 microns (e.g., the crystalline sodium salt of Compound A having a mean particle size of from about 1 to about 10 microns);
(bb1) the nonionic surfactant is a poloxamer or a polysorbate;
(bb2) the nonionic surfactant is a poloxamer (e.g., poloxamer 338 or poloxamer 407);
(cc1) the disintegrant is croscarmellose sodium, crospovidone, povidone, or sodium starch glycolate;
(cc2) the disintegrant is croscarmellose sodium;
(dd1) the encapsulated composition further comprises a lubricant, wherein the method further comprises lubricating the milled mixture from Step C with the lubricant prior to encapsulation in Step D;
(dd2) the encapsulated composition further comprises a diluent (e.g., lactose, microcrystalline cellulose, or both lactose and microcrystalline cellulose), a lubricant (e.g., magneisum stearate, stearic acid, sodium stearyl fumarate, or a combination of any two of the foregoing), a binder (e.g., hydroxypropyl cellulose), and optionally an antioxidant (e.g., BHA or no antioxidant); wherein the mixture employed in granulation Step A comprises Compound I (or II or A or B) or its salt, the nonionic surfactant, the disintegrant, the diluent, the binder, and the optional antioxidant; and wherein the method further comprises lubricating the milled mixture from Step C with the lubricant prior to encapsulation in Step D;
(dd3) the encapsulated composition further comprises a first diluent (e.g., lactose), a second diluent (e.g., microcrystalline cellulose), a lubricant (e.g., magnesium stearate, sodium stearyl fumarate, or a combination of magnesium stearate and sodium stearyl fumarate), and a binder (e.g., hydroxypropyl cellulose); wherein the mixture employed in granulation Step A comprises Compound I (or II or A or B) or its salt, the nonionic surfactant, the disintegrant, the first diluent, and the binder; and wherein the method further comprises blending the milled mixture of Step C with the second diluent, and then lubricating the milled and blended mixture with the lubricant prior to encapsulation in Step D;
(dd4) the encapsulated composition is as set forth in (dd2), wherein the encapsulated composition comprises from about 5 to about 40 wt. % Compound A sodium salt, from about 0.5 to about 15 wt. % poloxamer 407, from about 10 to about 40 wt. % lactose, from about 10 to about 40 wt. % microcrystalline cellulose, from about 0.5 to about 5 wt. % croscarmellose sodium, from about 0.2 to about 6 wt. % sodium stearyl fumarate, from about 0.1 to about 20 wt. % hydroxypropyl cellulose, and from 0 to about 0.1 wt. % (e.g., from about 0.01 to about 0.1 wt. %) BHA.
(ee1) in Step A, the mixture that is wet granulated is prepared by mixing an aqueous solution of the nonionic surfactant with a dry blend comprising Compound I (or II or A or B) or its salt, and disintegrant;
(ee2) the encapsulated composition further comprises a diluent and a binder; and in Step A, the mixture that is wet granulated is prepared by mixing an aqueous solution of the nonionic surfactant with a dry blend comprising Compound I (or II or A or B) or its salt, the diluent, the disintegrant, and the binder;
(ee3) in Step A, the mixture that is wet granulated is prepared by adding a granulating fluid comprising water, alcohol (e.g., C₁₋₄ alkyl alcohols such as ethanol), or an alcohol-water mixture (e.g., 5 to 95 wt. % ethanol-95 to 5 wt. % water solution, such as 95 wt. % ethanol-5 wt. % water) to a dry blend comprising Compound I (or II or A or B) or its salt, the nonionic surfactant, and the disintegrant; and
(ee4) the encapsulated composition further comprises a diluent and a binder; and in Step A, the mixture that is wet granulated is prepared by adding a granulating fluid comprising water, alcohol (e.g., C₁₋₄ alkyl alcohols such as ethanol), or an alcohol-water mixture (e.g., 5 to 95 wt. % ethanol-95 to 5 wt. % water solution, such as 95 wt. % ethanol-5 wt. % water) to a dry blend comprising Compound I (or II or A or B) or its salt, the nonionic surfactant, the diluent, the disintegrant, and the binder. (If an antioxidant is present, it is mixed with all or a portion of the granulating fluid before addition to the dry blend.)

The present invention also includes an encapsulated pharmaceutical composition prepared by the method comprising Steps A to D as just described.

The active drug substances employed in the pharmaceutical compositions of the present invention (e.g., the 8-hydroxy-1,6-naphthyridine-7-carboxamide compounds of Formula (I) and (II) and Compounds A and B) are inhibitors of HIV integrase. Representative compounds embraced by Formula I have been tested in an integrase inhibition assay in which strand transfer is catalyzed by recombinant integrase, and have been found to be active inhibitors of HIV integrase. Integrase inhibition activity can be determined, for example, using the assay described in Hazuda et al., J. Virol. 1997, 71: 7005-7011. Representative compounds have also been found to be active in an assay for the inhibition of acute H[V infection of T-lymphoid cells conducted in accordance with Vacca et al., Proc. Natl. Acad. Sci. USA 1994, 91: 4096-4100. In particular, Compound A and Compound B are each potent HIV integrase inhibitors in the strand transfer assay, and each is very effective in inhibiting HIV replication. Further description of representative compound embraced by Formula I, assays for measuring their integrase inhibition activity and HIV replication activity can be found in WO 02/30930.

The active drug substances in the pharmaceutical compositions of the present invention can be employed in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof). Suitable salts include acid addition salts which may, for example, be formed by mixing a solution of the compound with a solution of a pharmaceutically acceptable acid such as hydrochloric acid, sulfuric acid, acetic acid, trifluoroacetic acid, or benzoic acid. The compounds carry an acidic moiety (i.e., the hydroxy at the 8-position of the naphthyridine ring, also referred to herein as the “phenol”), so that suitable pharmaceutically acceptable salts thereof can include alkali metal salts (e.g., sodium or potassium salts), alkaline earth metal salts (e.g., calcium or magnesium salts), and salts formed with suitable organic ligands such as quaternary ammonium salts. Alkali metal salts (e.g., sodium salts) of the compounds can be formed by treating the compound dissolved in a suitable solvent with an aqueous solution of the alkali metal hydroxide (e.g., NaOH). The sodium salt of Compound A can be obtained, for example, by dissolving a monoethanolate of Compound A in an alcohol (e.g., methanol or ethanol) optionally in admixture with water as a co-solvent and treating the resulting solution with NaOH to form the sodium salt.

Particle size can affect the oral bioavailability of the active drug substances employed in the pharmaceutical compositions of the invention. For example, Compound A sodium salts milled to have a mean particle size of either about 3 μm (90% less than 5 μm) or about 6 μm (90% less than 10 μm) have exhibited improved oral bioavailabilities when administered to animals, relative to the unmilled salts having a mean particle size of about 40 μm (90% less than 100 μm). The milled Compound A Na salt has also exhibited better mechanical strength for tablet formulation than the unmilled salt. Accordingly, embodiments of the present invention include each of the pharmaceutical compositions defined and described above, wherein the active drug substance (i.e., Compound I or II or A or B or a pharmaceutically acceptable salt thereof) employed therein has a mean particle size of from about 1 to about 20 μm, or from about 1 to about 10 μm (e.g., from about 3 to about 6 μm wherein 90% of the particles are less than 10 μm).

The pharmaceutical compositions of the present invention are useful in the inhibition of HIV integrase, the prevention or treatment of infection by HIV and the prevention, treatment or the delay in the onset of consequent pathological conditions such as AIDS. Preventing AIDS, treating AIDS, delaying the onset of AIDS, or preventing or treating infection by HIV is defined as including, but not limited to, treatment of a wide range of states of HIV infection: AIDS, AR^{c}, both symptomatic and asymptomatic, and actual or potential exposure to HIV. For example, the compositions of this invention are useful in treating infection by HIV after suspected past exposure to HIV by such means as blood transfusion, exchange of body fluids, bites, accidental needle stick, or exposure to patient blood during surgery.

The present invention includes a method for inhibiting HIV integrase in a subject in need thereof which comprises administering to the subject the pharmaceutical composition of the present invention as originally defined above. The invention also includes a method for preventing or treating HIV infection or for preventing, treating or delaying the onset of AIDS in a subject in need of such treatment, which comprises administering to the subject the pharmaceutical composition of the present invention as originally defined above. In these methods, the compositions of the present invention can optionally be employed in combination with one or more HIV/AIDS treatment agents selected from HIV/AIDS antiviral agents, anti-infective agents, and immunomodulators. Embodiments of these methods include the methods as just described wherein the pharmaceutical composition is a pharmaceutical composition as set forth in any one of the foregoing embodiments or in an aspect thereof.

The present invention also includes the pharmaceutical composition of the present invention as originally defined (i) for use in, (ii) for use as a medicament for, or (iii) for use in the preparation of a medicament for: (a) inhibiting HIV protease, (b) preventing or treating infection by HIV, or (c) preventing, treating or delaying the onset of AIDS. In these uses, the compounds of the present invention can optionally be employed in combination with one or more HIV/AIDS treatment agents selected from HIV/AIDS antiviral agents, anti-infective agents, and immunomodulators. Embodiments of these uses include the uses as just described wherein the pharmaceutical composition is a pharmaceutical composition as set forth in one of the foregoing embodiments or in an aspect thereof.

The pharmaceutical compositions of this invention are administered orally in a suitable dosage form, such as capsules or compressed tablets. The compositions can be administered in a dosage range of from about 0.001 to about 1000 mg/kg of mammal (e.g., human) body weight per day in a single dose or in divided doses. One preferred dosage range is from about 0.01 to about 500 mg/kg body weight per day orally in a single dose or in divided doses. Another preferred dosage range is from about 0.1 to about 100 mg/kg body weight orally in single or divided doses. For oral administration, the compositions can suitably be provided in the form of tablets or capsules containing from about 1 to about 1000 milligrams of the active ingredient, particularly 1, 5, 10, 15, 20, 25, 50, 75, 100, 150, 200, 250, 300, 400, 500, 600, and 800 milligrams of the active ingredient for the symptomatic adjustment of the dosage to the patient to be treated. A preferred oral dose is an encapsulated or tabletted composition of the invention containing from about 5 to about 500 mg of active ingredient. In another preferred dose, the encapsulated or tabletted composition contains from about 10 to about 100 mg of active ingredient. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy. As noted earlier, although the active drug substance (i.e., active ingredient) can be administered as a salt, all references herein to the amount of active ingredient are to the free acid (alternatively referred to herein as the “free phenol”) or free base form of the compound, unless expressly stated to the contrary in a particular context.

As noted above, the present invention is also directed to use of the pharmaceutical compositions of the present invention with one or more agents useful in the treatment of HIV infection or AIDS. For example, the compositions of this invention may be effectively administered, whether at periods of pre-exposure and/or post-exposure, in combination with effective amounts of one or more of the HIV/AIDS antivirals, imunomodulators, antiinfectives, or vaccines useful for treating HIV infection or AIDS. Suitable agents include the following HIV antivirals:

| | Manufacturer | |
|---|---|---|
| | (Tradename and/or | |
| Drug Name | Location) | Indication (Activity) |
| abacavir | Glaxo Welcome | HIV infection, AIDS, ARC |
| GW 1592 | (ZIAGEN ®) | (nucleoside reverse |
| 1592U89 | | transcriptase inhibitor) |
| abacavir + | GlaxoSmithKline | HIV infection, AIDS, ARC |
| lamivudine + zidovudine | (TRIZIVIR ®) | (nucleoside reverse |
| | | transcriptase inhibitors) |
| acemannan | Carrington Labs | ARC |
| | (Irving, TX) | |
| ACH 126443 | Achillion Pharm. | HIV infections, AIDS, ARC |
| | | (nucleoside reverse |
| | | transcriptase inhibitor) |
| acyclovir | Burroughs Wellcome | HIV infection, AIDS, ARC, |
| | | in combination with AZT |
| AD-439 | Tanox Biosystems | HIV infection, AIDS, ARC |
| AD-519 | Tanox Biosystems | HIV infection, AIDS, ARC |
| adefovir dipivoxil | Gilead | HIV infection, AIDS, ARC |
| GS 840 | | (reverse transcriptase |
| | | inhibitor) |
| AL-721 | Ethigen | ARC, PGL, HIV positive, |
| | (Los Angeles, CA) | AIDS |
| alpha interferon | GlaxoSmithKline | Kaposi's sarcoma, HIV, in |
| | | combination w/Retrovir |
| AMD3100 | AnorMed | HIV infection, AIDS, |
| | | ARC |
| | | (CXCR4 antagonist) |
| amprenavir | GlaxoSmithKline | HIV infection, AIDS, |
| 141 W94 | (AGENERASE ®) | ARC (PI) |
| GW 141 | | |
| VX478 (Vertex) | | |
| ansamycin | Adria Laboratories | ARC |
| LM 427 | (Dublin, OH) | |
| | Erbamont | |
| | (Stamford, CT) | |
| antibody which neutralizes | Advanced Biotherapy | AIDS, ARC |
| pH labile alpha aberrant | Concepts (Rockville, | |
| interferon | MD) | |
| AR177 | Aronex Pharm | HIV infection, AIDS, ARC |
| atazanavir (BMS 232632) | Bristol-Myers Squibb | HIV infection, AIDS, ARC |
| | (ZRIVADA ®) | (protease inhibitor) |
| beta-fluoro-ddA | Nat'l Cancer Institute | AIDS-associated diseases |
| BMS-232623 | Bristol-Myers Squibb/ | HIV infection, AIDS, |
| (CGP-73547) | Novartis | ARC |
| | | (protease inhibitor) |
| BMS-234475 | Bristol-Myers Squibb/ | HIV infection, AIDS, |
| (CGP-61755) | Novartis | ARC (protease inhibitor) |
| capravirine | Pfizer | HIV infection, AIDS, |
| (AG-1549, S-1153) | | ARC (non-nucleoside |
| | | reverse transcriptase |
| | | inhibitor) |
| CI-1012 | Warner-Lambert | HIV-1 infection |
| cidofovir | Gilead Science | CMV retinitis, herpes, |
| | | papillomavirus |
| curdlan sulfate | AJI Pharma USA | HIV infection |
| cytomegalovirus immune | MedImmune | CMV retinitis |
| globin | | |
| cytovene | Syntex | sight threatening CMV |
| ganciclovir | | peripheral CMV |
| | | retinitis |
| delavirdine | Pharmacia-Upjohn | HIV infection, AIDS, |
| | (RESCRIPTOR ®) | ARC (non-nucleoside |
| | | reverse transcriptase |
| | | inhibitor) |
| dextran Sulfate | Ueno Fine Chem. Ind. | AIDS, ARC, HIV |
| | Ltd. (Osaka, Japan) | positive asymptomatic |
| ddC | Hoffman-La Roche | HIV infection, AIDS, ARC |
| (zalcitabine, | (HIVID ®) | (nuclesodie reverse |
| dideoxycytidine) | | transcriptase inhibitor) |
| ddI | Bristol-Myers Squibb | HIV infection, AIDS, ARC; |
| dideoxyinosine | (VIDEX ®) | combination with AZT/d4T |
| | | (nucleoside reverse |
| | | transcriptase inhibitor) |
| DPC 681 & DPC 684 | DuPont | HIV infection, AIDS, ARC |
| | | (protease inhibitors) |
| DPC 961 & DPC 083 | Bristol-Myers Squibb | HIV infection AIDS, ARC |
| | (from DuPont Pharma) | (non-nucleoside reverse |
| | | transcriptase inhibitors) |
| EL10 | Elan Corp, PLC | HIV infection |
| | (Gainesville, GA) | |
| efavirenz | Bristol-Myers Squibb | HIV infection, AIDS, |
| (DMP 266) | (SUSTIVA ®) | ARC (non-nucleoside RT |
| | Merck (STOCRIN ®) | inhibitor) |
| famciclovir | Novartis | herpes zoster, herpes |
| | (FAMVIR ®) | simplex |
| emtricitabine | Gilead (from Triangle | HIV infection, AIDS, ARC |
| FTC | Pharmaceuticals) | (nucleoside reverse |
| | (COVIRACIL ®) | transcriptase inhibitor) |
| | Emory University | |
| emvirine | Gilead (from Triangle | HIV infection, AIDS, ARC |
| | Pharmaceuticals) | (non-nucleoside reverse |
| | (COACTINON ®) | transcriptase inhibitor) |
| enfuvirtide | Trimeris & Roche | HIV infection, AIDS, ARC |
| T-20 | (FUZEON ®) | (fusion inhibitor) |
| HBY097 | Hoechst Marion Roussel | HIV infection, AIDS, ARC |
| | | (non-nucleoside reverse |
| | | transcriptase inhibitor) |
| fosamprenavir | Glaxo Smith Kline | HIV infection, AIDS, ARC |
| | | (prodrug of the PI |
| | | amprenavir) |
| hypericin | VIMRx Pharm. | HIV infection, AIDS, ARC |
| recombinant human | Triton Biosciences | AIDS, Kaposi's sarcoma, |
| interferon beta | (Almeda, CA) | ARC |
| interferon alfa-n3 | Interferon Sciences | ARC, AIDS |
| indinavir | Merck (CRIXIVAN ®) | HIV infection, AIDS, ARC, |
| | | asymptomatic HIV positive, |
| | | also in combination with |
| | | AZT/ddI/ddC |
| ISIS 2922 | ISIS Pharmaceuticals | CMV retinitis |
| JE2147/AG1776 | Agouron | HIV infection, AIDS, ARC |
| | | (protease inhibitor) |
| KNI-272 | Nat'l Cancer Institute | HIV-assoc. diseases |
| lamivudine, 3TC | GlaxoSmithKline | HIV infection, AIDS, |
| | (EPIVIR ®) | ARC (nucleoside reverse |
| | | transcriptase inhibitor); |
| | | also with AZT |
| lobucavir | Bristol-Myers Squibb | CMV infection |
| lopinavir (ABT-378) | Abbott | HIV infection, AIDS, ARC |
| | | (protease inhibitor) |
| lopinavir + ritonavir | Abbott (KALETRA ®) | HIV infection, AIDS, ARC |
| (ABT-378/r) | | (protease inhibitor) |
| mozenavir | AVID (Camden, NJ) | HIV infection, AIDS, ARC |
| (DMP-450) | | (protease inhibitor) |
| nelfinavir | Agouron | HIV infection, AIDS, |
| | (VIRACEPT ®) | ARC (protease inhibitor) |
| nevirapine | Boeheringer | HIV infection, AIDS, |
| | Ingleheim | ARC (non-nucleoside |
| | (VIRAMUNE ®) | reverse transcriptase |
| | | inhibitor) |
| novapren | Novaferon Labs, Inc. | HIV inhibitor |
| | (Akron, OH) | |
| peptide T | Peninsula Labs | AIDS |
| octapeptide | (Belmont, CA) | |
| sequence | | |
| PRO 140 | Progenics | HIV infection, AIDS, ARC |
| | | (CCR5 co-receptor inhibitor) |
| PRO 542 | Progenics | HIV infection, AIDS, ARC |
| | | (attachment inhibitor) |
| trisodium | Astra Pharm. Products, | CMV retinitis, HIV infection, |
| phosphonoformate | Inc | other CMV infections |
| PNU-140690 | Pharmacia Upjohn | HIV infection, AIDS, ARC |
| | | (protease inhibitor) |
| probucol | Vyrex | HIV infection, AIDS |
| RBC-CD4 | Sheffield Med. Tech | HIV infection, AIDS, |
| | (Houston TX) | ARC |
| ritonavir | Abbott | HIV infection, AIDS, |
| (ABT-538) | (RITONAVIR ®) | ARC (protease inhibitor) |
| saquinavir | Hoffmann-LaRoche | HIV infection, AIDS, |
| | (FORTOVASE ®) | ARC (protease inhibitor) |
| stavudine; d4T | Bristol-Myers Squibb | HIV infection, AIDS, ARC |
| didehydrodeoxy- | (ZERIT ®) | (nucleoside reverse |
| thymidine | | transcriptase inhibitor) |
| T-1249 | Trimeris | HIV infection, AIDS, ARC |
| | | (fusion inhibitor) |
| TAK-779 | Takeda | HIV infection, AIDS, ARC |
| | | (injectable CCR5 receptor |
| | | antagonist) |
| tenofovir | Gilead (VIREAD ®) | HIV infection, AIDS, ARC |
| | | (nucleotide reverse |
| | | transcriptase inhibitor) |
| tipranavir (PNU-140690) | Boehringer Ingelheim | HIV infection, AIDS, ARC |
| | | (protease inhibitor) |
| TMC-120 & TMC-125 | Tibotec | HIV infections, AIDS, ARC |
| | | (non-nucleoside reverse |
| | | transcriptase inhibitors) |
| TMC-126 | Tibotec | HIV infection, AIDS, ARC |
| | | (protease inhibitor) |
| valaciclovir | GlaxoSmithKline | genital HSV & CMV |
| | | infections |
| virazole | Viratek/ICN (Costa | asymptomatic HIV positive, |
| ribavirin | Mesa, CA) | LAS, ARC |
| zidovudine; AZT | GlaxoSmithKline | HIV infection, AIDS, ARC, |
| | (RETROVIR ®) | Kaposi's sarcoma in |
| | | combination with other |
| | | therapies (nucleoside reverse |
| | | transcriptase inhibitor) |

Other antivirals plus immunomodulators, antiinfectives, or vaccines useful for treating HIV infection or treating or delaying the onset of AIDS that can be used in combination with the pharmaceutical compositions of the present invention are disclosed in Table 1 of WO 01/38332, which is herein incorporated by reference in its entirety. It will be understood that the scope of combinations of compositions of this invention with HIV/AIDS antivirals, immunomodulators, anti-infectives or vaccines is not limited to those listed above and those disclosed in the above-referenced Table in WO 01/38332, but includes in principle any combination with any other pharmaceutical composition useful for the treatment of AIDS. The HIV/AIDS antivirals and other agents will typically be employed in these combinations in their conventional dosage ranges and regimens as reported in the art, including the dosages described in the Physicians' Desk Reference, 54^{th} edition, Medical Economics Company, 2000. The dosage ranges of the active drug substances in the pharmaceutical compositions of the present invention employed in these combinations are suitably the same as those set forth above.

The term “administration” and variants thereof (e.g., “administering” a pharmaceutical composition) in reference to the pharmaceutical composition of the invention means providing the composition to the individual in need of treatment. When the composition of the invention is provided in combination with one or more other active agents (e.g., antiviral agents useful for treating HIV infection or AIDS), “administration” and its variants are each understood to include concurrent and sequential provision of the composition and other agents. Thus, for example, administration of the pharmaceutical composition of the invention in combination with another antiviral agent means that the composition of the invention can be administered before, at the same time, or after the other agent.

The term “subject” as used herein refers to an animal, preferably a mammal, most preferably a human, who has been the object of treatment, observation or experiment.

The term “therapeutically effective amount” as used herein means that amount of active compound or pharmaceutical agent that elicits the biological or medicinal response in a tissue, system, animal or human that is being sought by a researcher, veterinarian, medical doctor or other clinician, which includes alleviation of the symptoms of the disease being treated.

The term “C₁₋₆ alkyl” (or “C₁-C₆ alkyl”) means linear or branched chain alkyl groups having from 1 to 6 carbon atoms and includes all of the hexyl alkyl and pentyl alkyl isomers as well as n-, iso-, sec- and t-butyl, n- and isopropyl, ethyl and methyl. “C₁₋₄ alkyl” means n-, iso-, sec- and t-butyl, n- and isopropyl, ethyl and methyl.

The term “—C₁₋₆ alkyl-” refers to a C₁ to C₆ linear or branched alkyl group as just defined which is bivalent. It can alternatively be referred to as “C₁₋₆ alkylene” or “C₁₋₆ alkanediyl”. A class of alkylenes of particular interest with respect to the invention is —(CH₂)₁₋₆—, and sub-classes of particular interest include —(CH₂)₁₋₄—, —(CH₂)₁₋₃—, —(CH₂)₁₋₂—, and —CH₂—.

The term “C₃₋₆ cycloalkyl” (or “C₃-C₆ cycloalkyl”) means a cyclic ring of an alkane having three to six total carbon atoms (i.e., cyclopropyl, cyclobutyl, cyclopentyl, or cyclohexyl).

The term “halogen” (or “halo”) refers to fluorine, chlorine, bromine and iodine (alternatively referred to as fluoro, chloro, bromo, and iodo).

The term “C₁₋₆ haloalkyl” (which may alternatively be referred to as “C₁-C₆ haloalkyl” or “halogenated C₁-C₆ alkyl”) means a C₁ to C₆ linear or branched alkyl group as defined above with one or more halogen substituents. The term “C₁₋₄ haloalkyl” has an analogous meaning. The term “C₁₋₆fluoroalkyl” has an analogous meaning except that the halogen substituents are restricted to fluoro. A class of fluoroalkyls of particular interest with respect to the invention is the series (CH₂)₀₋₄CF₃ (i.e., trifluoromethyl, 2,2,2-trifluoroethyl, 3,3,3-trifluoro-n-propyl, etc.).

The term “saturated heterocyclic ring” refers to a 5- to 7-membered saturated monocyclic ring which consists of carbon atoms and one or more (e.g., from 1 to 4) heteroatoms independently selected from N, O and S. Representative examples include piperidinyl, piperazinyl, azepanyl (i.e., 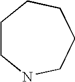
), pyrrolidinyl, pyrazolidinyl, imidazolidinyl, oxazolidinyl, isoxazolidinyl, morpholinyl, thiomorpholinyl, thiazolidinyl, isothiazolidinyl, tetrahydrothienyl, tetrahydrofuryl (or tetrahydrofuranyl), thiazinanyl (e.g., 1,2-thiazinanyl 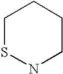
), thiadiazinanyl (e.g., 1,2,6-thiadiazinanyl 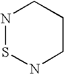
), dioxanyl, hexahydropyrirnidinyl, thiazepanyl, thiadiazepanyl, dithiazepanyl, and diazepanyl. The term does not include any saturated heterocyclic rings that are chemically unstable or chemically not allowed.

The term “heteroaromatic ring” refers a 5- or 6-membered monocyclic aromatic ring which consists of carbon atoms and one or more (e.g., from 1 to 4) heteroatoms independently selected from N, O and S. Representative examples of heteroaromatic rings include pyridyl, pyrrolyl, pyrazinyl, pyrimidinyl, pyridazinyl, thienyl (or thiophenyl), thiazolyl, furanyl, imidazolyl, pyrazolyl, triazolyl, tetrazolyl, oxazolyl, isooxazolyl, oxadiazolyl, thiazolyl, isothiazolyl, and thiadiazolyl. The term does not include any heteroaromatic rings that are chemically unstable or chemically not allowed.

Unless expressly stated to the contrary, all ranges cited herein are inclusive. For example, a saturated heterocyclic ring described as containing from “1 to 4 heteroatoms” means the heterocycle can contain 1, 2, 3 or 4 heteroatoms.

When any variable (e.g., R^{a}, R^{b}, or R^{c}) occurs more than one time in any constituent or in Formula I or in any other formula depicting and describing compounds employed in the pharmaceutical compositions of the invention, its definition on each occurrence is independent of its definition at every other occurrence. Also, combinations of substituents and/or variables are permissible only if such combinations result in stable compounds.

The term “substituted” (e.g., as in “a saturated heterocyclic ring which is optionally substituted with from 1 to 4 substituents . . . ”) includes mono- and polysubstitution by a named substituent to the extent such single and multiple substitution (including multiple substitution at the same site) is chemically allowed.

To the extent that the active drug substances in the pharmaceutical compositions of the present invention can have asymmetric centers, except when specifically noted, the active drug substance can consist of mixtures of stereoisomers or as individual diastereomers, or enantiomers.

Abbreviations used in the instant specification, particularly the Schemes and Examples, include the following:
AIDS=acquired immunodeficiency syndrome
APCI=atmospheric pressure chemical ionization mass spectroscopy
ARC=AIDS related complex
BOC or Boc=t-butyloxycarbonyl
BOP=benzotriazol-1-yloxytris-(dimethylamino)phosphonium hexafluorophosphate
n-BuLi=n-butyllithium
DEAD=diethylazodicarboxylate
DIPA=diisopropylamine
DIPEA=diisopropylethylamine
DMF=dimethylformamide
DMPU=1,3-dimethyl-3,4,5,6-tetrahydro-2(1H)-pyrimidinone
DMSO=dimethyl sulfoxide
dppf=1,1′-bis(diphenylphosphino)ferrocene
EDC or EDAC=1-ethyl-3-(3-dimethylaminopropyl)carbodiimide
ES-MS=eletron spray mass spectroscopy
Et=ethyl
EtOAc=ethyl acetate
HIV=human immunodeficiency virus
HOAt=1-hydroxy-7-azabensotriazole
HOBt=1-hydroxy benzotriazole hydrate
HPLC=high performance liquid chromatography
HRMS=high resolution mass spectroscopy
LC=liquid chromatography
Me=methyl
MeOH=methanol
Ms=mesyl or methanesulfonyl
MS=mass spectroscopy
MTBE=methyl tert-butyl ether
NBS=N-bromosuccinimide
NIS=N-iodosuccinimide
NMM=N-methyl morpholine
NMR=nuclear magnetic resonance
Ph=phenyl
PMBCl=p-methoxybenzyl chloride
Pr=propyl
SLS=sodium lauryl sulfate
TEA=triethylamine
Tf₂O=triflic anhydride
TFA=trifluoroacetic acid
THF=tetrahydrofuran
TLC=thin layer chromatography
TsCl=toluenesulfonyl chloride
UV=ultraviolet

The active drug substances in the pharmaceutical compositions of the present invention (i.e., Compound I, Compound II, Compound A, and Compound B) can be prepared according to the following reaction schemes and examples, or modifications thereof, using readily available starting materials, reagents and conventional synthesis procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples. Unless otherwise indicated, all variables are as defined above.

The compounds of the present invention can be prepared by the coupling of suitable 1,6-naphthyridine-7-carboxylic acids (or acid derivatives such as acid halides or esters) with the appropriate benzylamines. Scheme 1 depicts the coupling reaction.

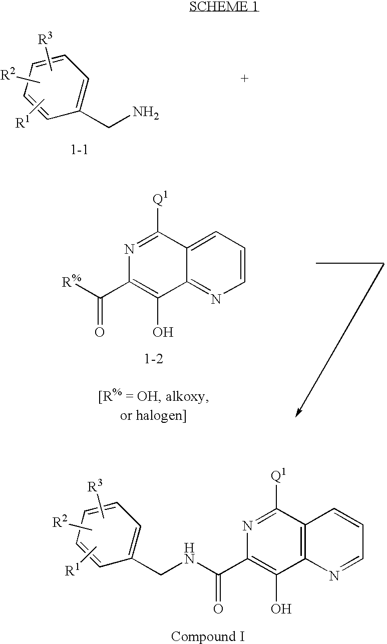

Methods for coupling carboxylic acids with amines to form carboxamides are well known in the art. Suitable methods are described, for example, in Jerry March, Advanced Organic Chemistry, 3rd edition, John Wiley & Sons, 1985, pp. 370-376, or in M. Bodanszky, The Practice of Peptide Synthesis, Springer-Verlag, 1984. Amines of formula 1-1 can be prepared, for example, by the reaction of a suitable benzyl halide with ammonia, by conversion of a suitable benzyl halide with hexamethylenetetramine, by treating the halide with potassium phthalimide and hydrolyzing the product, and by converting a benzyl halide to an azide and then reducing the azide to an amine; which methods are described, for example, in Jerry March, Advanced Organic Chemistry, 3^{rd} edition, John Wiley & Sons, 1985, pp. 364-365, 366, 377-378, 380, and 1106. Amines of formula 1-1 can also be prepared using, for example, the methods described in Richard Larock, Comprehensive Organic Transformations, 2^{nd} edition, Wiley-VCH Publishers Inc, 1999, pp 753-879, or routine variations thereof. Naphthyridine carboxylic acids of formula 1-2 can be prepared using methods described in Ochiai et al., Chem. Ber. 1937, 70: 2018, 2023; and Albert et al., J. Chem. Soc. 1952, 4985, 4991; or routine variations thereof. The schemes set forth below illustrate and expand upon the chemistry portrayed in Scheme 1.

Scheme 2 depicts a method for preparing benzylamine reactants having at least one ortho-aminocarbonyl group on the benzyl ring. Substituted toluene 2-1 is functionalized on the methyl group via radical bromination to give the bromide 2-2. Radical brominations are well known in the art and are described, for example, in J. March, Advanced Organic Chemistry, 3rd edition, John Wiley & Sons, 1985, p. 625. The azide 2-3 can then be obtained by displacement of the bromide with azide (see J. March, Advanced Organic Chemistry, 3rd edition, John Wiley & Sons, 1985, p. 380), followed by reduction of the azide using triphenylphosphine and water to afford the amine 2-4. Similar reductions are described in Tetrahedron 2000, 56(52): 10175-10184; in J. Am. Chem. Soc. 2001, 123(5): 875-885; and in Zhou, Tett Lett. 1999, 40: 2729. Following protection of the amino group on 2-4 using BOC, the iodide can be transformed into the carboxyamide 2-6 through a palladium-catalyzed carbonylation reaction in the presence of a suitable amine, in a manner similar to that described in G. Ortar, Tett. Lett. 1986, 27: 3931. Following removal of the BOC group, amine 2-7 can be coupled to a suitable naphthyridine carboxylic acid, e.g., with EDC and HOAt in the presence of a suitable base such as triethylamine.

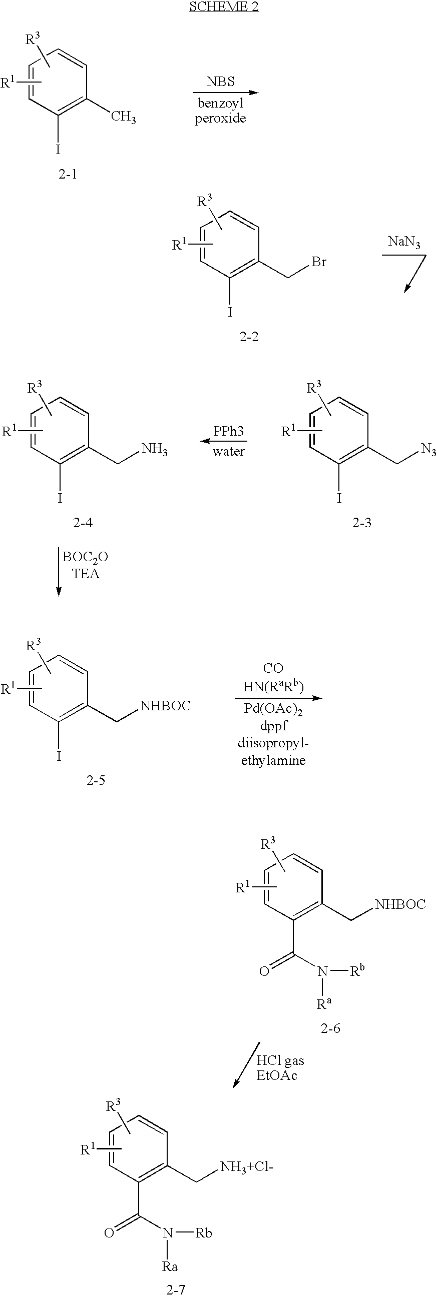

In Scheme 3, following the procedure set forth in Ornstein et al., J. Med. Chem. 1989, 32: 827-833, quinolinic anhydride 3-1 can be opened with isopropanol to provide mono acid 3-2, which can be converted to the corresponding acyl chloride 3-3 (e.g., by refluxing thionyl chloride). Acyl chloride 3-3 can then be reduced (e.g., with NaBH₄ or LiBH₄) to the corresponding alcohol 3-4, which can be converted to the corresponding bromide through the action of bromine in the presence of triphenylphosphine. Alkylation of the bromide with the sodium anion of phenylsulfonamide 3-5 in a polar aprotic solvent like DMF can provide sulfonamide 3-6, which can be treated with a base (e.g., alkali metal alkoxide such as sodium methoxide) to provide the bicyclic ester 3-7 via a Dieckmann cyclization. Saponification of the ester (e.g., with aqueous NaOH at reflux) will afford the acid 3-8. The acid 3-8 can be activated with triphosgene and coupled with a variety of benzylamines to provide the compounds of the invention 3-9.

The starting anhydrides of formula 3-1 can be prepared via methods described in Philips et al., Justus Liebigs Ann. Chem. 1895, 288: 2535; Bernthsen et al., Chem. Ber. 1887; 20: 1209; Bly et al., J. Org. Chem. 1964, 29: 2128-2135; and Krapcho et al., J. Heterocycl. Chem. 1993, 30: 1597-1606; or routine variations thereof.

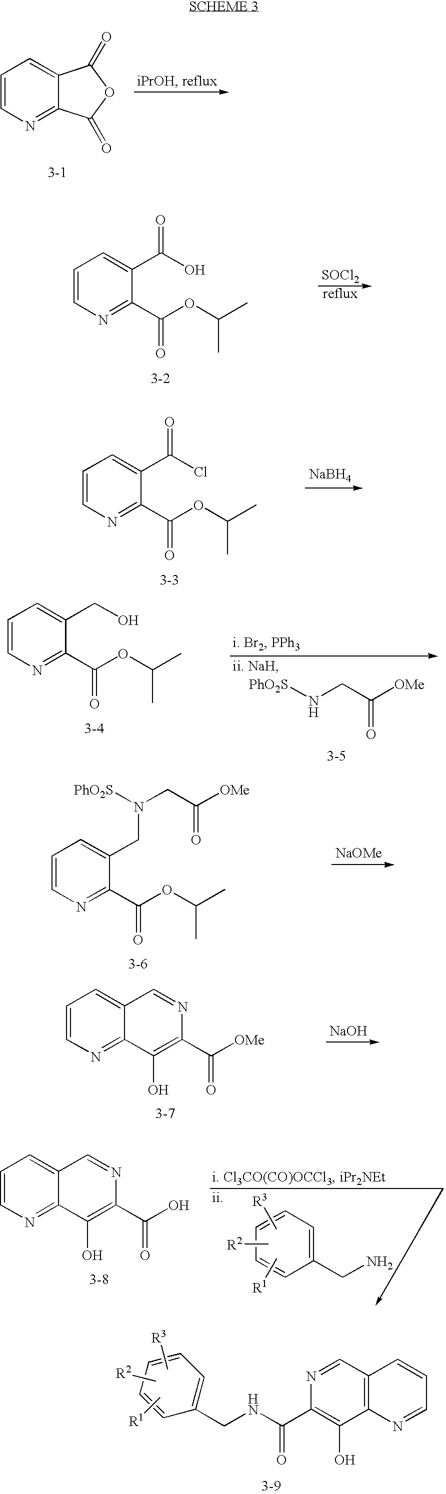

Scheme 4 depicts an alternative synthesis in which alcohol 3-4 can undergo the Mitsunobu reaction with the phenylsulfonamide of glycine methyl ester to provide 4-1. The sulfonamide 4-1 can again be elaborated to provide the acid 3-8, which can be coupled with a variety of amines using standard reagents to provide the compounds of the invention 3-9.

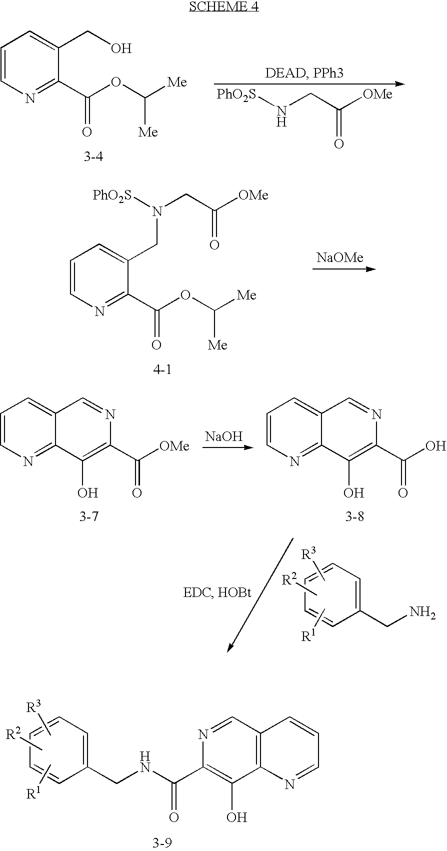

Scheme 5 depicts a variation of the synthesis shown in Scheme 4, wherein the acid 5-2 is reacted with ethyl chloroformate to form the mixed anhydride 5-3, which is reduced to alcohol 5-4. Alcohol 5-4 can undergo the Mitsunobu reaction with methyl tosylglycine to form the ester 5-5, which under treatment with base cyclizes to form the 1,6-naphthyridine 5-6. Bromination then yields the bromoester 5-7, which can be used as an intermediate in the preparation of compounds of Formula (I) and (II).

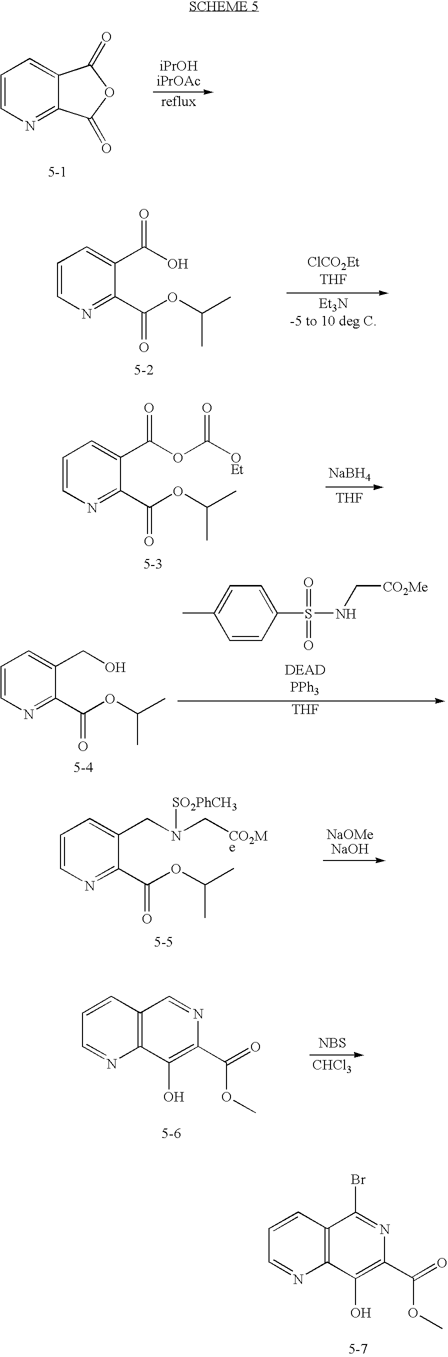

The preparation of compounds that feature additional substituents can be achieved in accordance with Scheme 6. Oxidation of the alcohol 3-4 with manganese dioxide in an inert solvent such as methylene chloride will provide aldehyde 6-1. The addition of Grignard reagents to aldehyde moiety 6-1 can occur regioselectively to provide the alcohol 6-2, which can then be elaborated to the compounds of the invention 6-6.

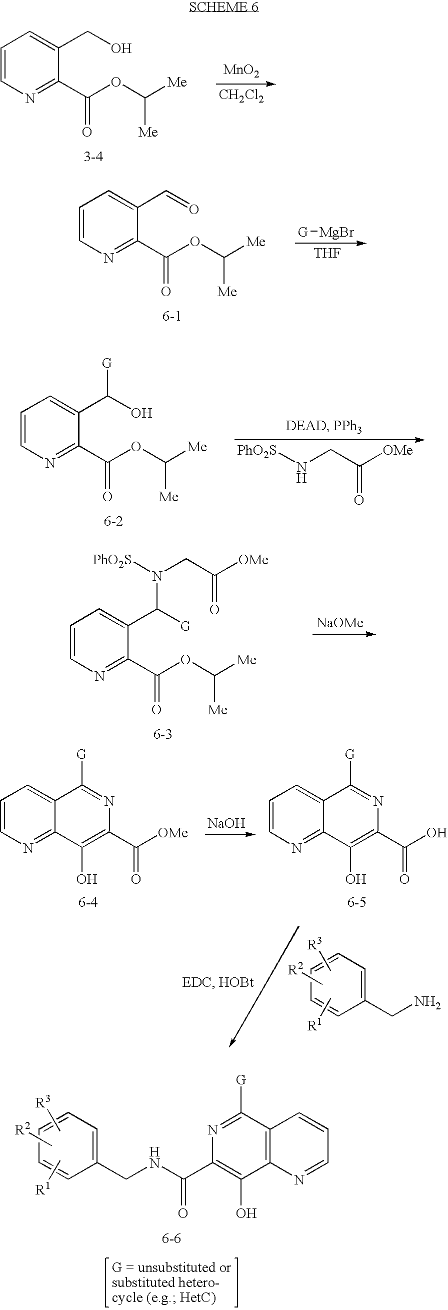

A further synthetic route to prepare compounds that are employed in pharmaceutical compositions of the invention is shown in Scheme 7. This methodology allows access to naphthyridine derivatives that are substituted at the 5 position. Briefly, a 2-substituted 5-hydroxypyridine derivative 7-1 can be treated with bromine to undergo bromination at the 6 position to afford 7-2, which can be converted to the methoxypyridine 7-3 and then oxidized to the corresponding N-oxide 7-4. The N-oxide can be nitrated to provide 7-5. Reduction of 7-5 with iron in the presence of ammonium chloride can provide the aniline 7-6, which can be reacted with an alpha,beta-unsaturated aldehyde or ketone in the presence of an acid catalyst like sulfuric acid to provide 7-7 via an annulation. The bromide 7-7 can be elaborated to the amide 7-9 via a sequence of carbonylation and amidation reactions.

2-Substituted 5-hydroxypyridine derivatives of formula 7-1 can be prepared via methods described in Sorm et al., Collect. Czech. Chem. Commun. 1949, 14: 331, 342; and Saksena et al., Tetrahedron Lett. 1993, 34: 3267-3270: or routine variations thereof.

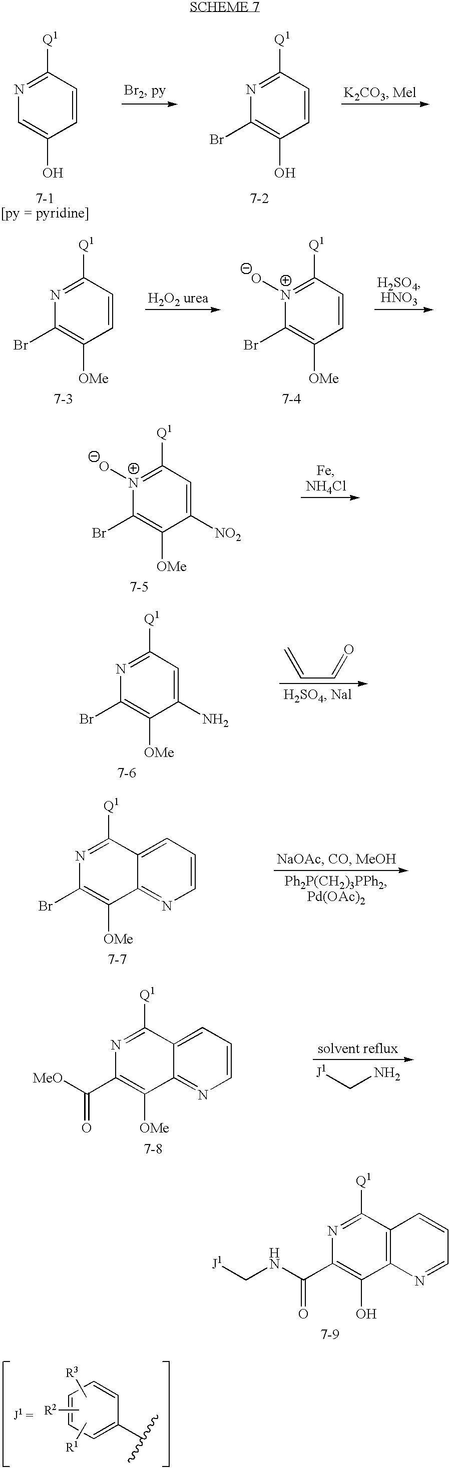

Preparation of compounds of the invention substituted with a sulfonamide can be prepared according to Scheme 8. The preparation includes halogenation of alkyl 8-hydroxy-naphthyridine carboxylate 8-1 with a halogenation agent such as N-bromosuccinimide, and then condensing the 5-halo-8-hydroxy-naphthyridine carboxylic ester 8-2 with sulfonamide 8-3 at elevated temperature (e.g., about 120° C.) in the presence of a copper promoter (e.g., copper(I) oxide) to afford sulfonamidonaphthyridine 8-4. The 7-position ester can then be hydrolyzed and the benzylamine portion attached through standard amide bond formation methods to give desired product 8-6.

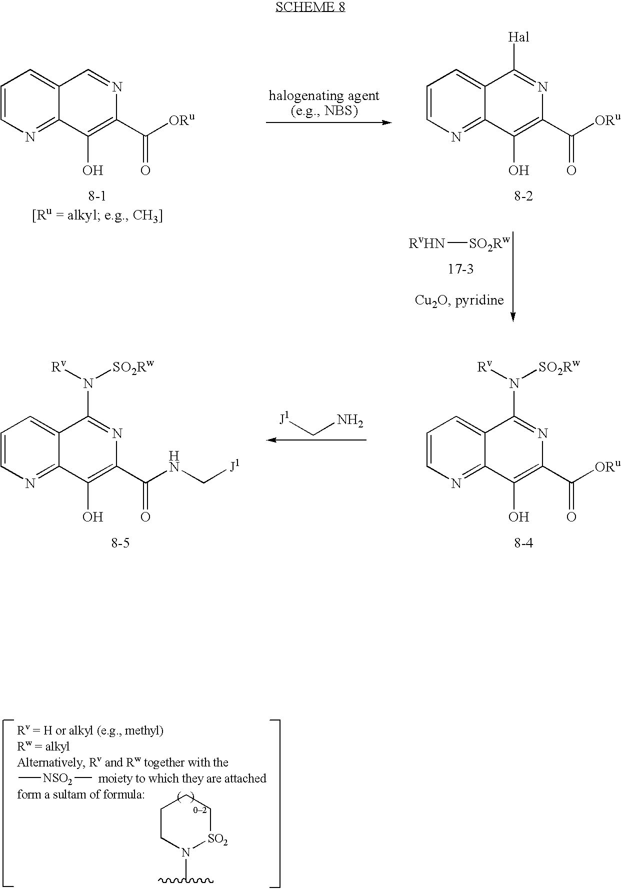

Scheme 9 shows a method for preparing compounds of the invention in which the benzylamine moiety has an ortho-substituted amino-2-oxoethyl group. In this scheme, amine 9-1 (commercially available) is coupled with a suitable naphthyridine carboxylic acid under standard EDC/HOAt coupling conditions in the presence of a suitable base (e.g., NMM) to afford amide product 9-2. The resulting ester can then be hydrolyzed to the acid which can then be coupled with a suitable amine.

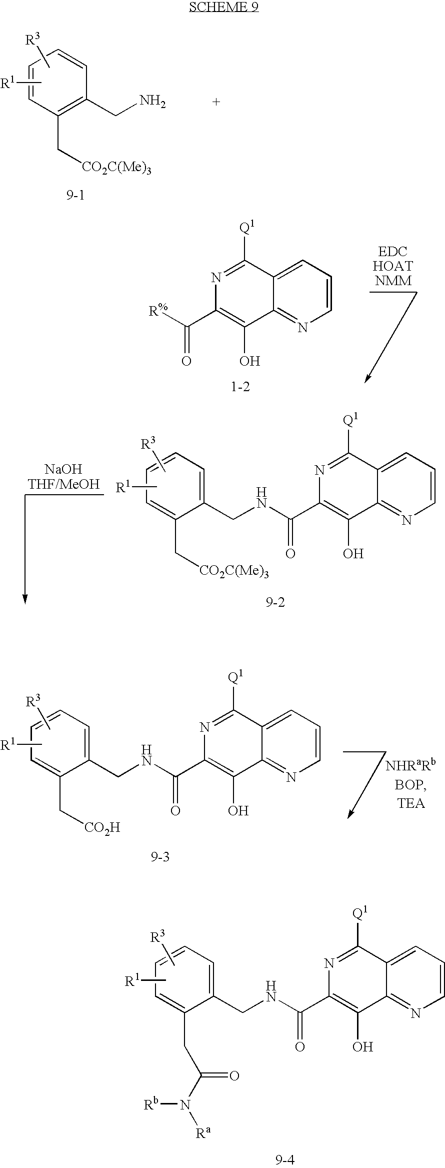

Scheme 10 describes the preparation of compounds having an aminocarboxy group at the 5-position of the naphthyridine ring. In this scheme, the brominated naphthyridine 10-1 is treated with carbon monoxide and methanol under palladium catalysis utilizing 1,1″-bis(diphenylphosphino)ferrocene as a ligand, using conditions similar to those described in Ortar, Tett. Letters 1986, 27 (33): 3931, to afford acylated naphthyridine 10-2. Removal of the tosyl protecting group with sodium methoxide in an alcholic solvent (e.g., trifluoroethanol) affords the dimethyl dicarboxylate 10-3, which can be selectively hydrolyzed under aqueous base conditions (e.g., as described in Jerry March, Advanced Organic Chemistry, 3rd edition, John Wiley & Sons, 1985, pp. 334-338) to the carboxylic acid 10-4. The amide 10-5 can then be obtained from 10-4 with conventional amide coupling reagents like BOP or EDC in the presence of excess amine. The 7-position ester can hen be hydrolyzed with aqueous base to afford the acid 10-6, which can then be coupled with a suitable benzylamine to give 10-7.

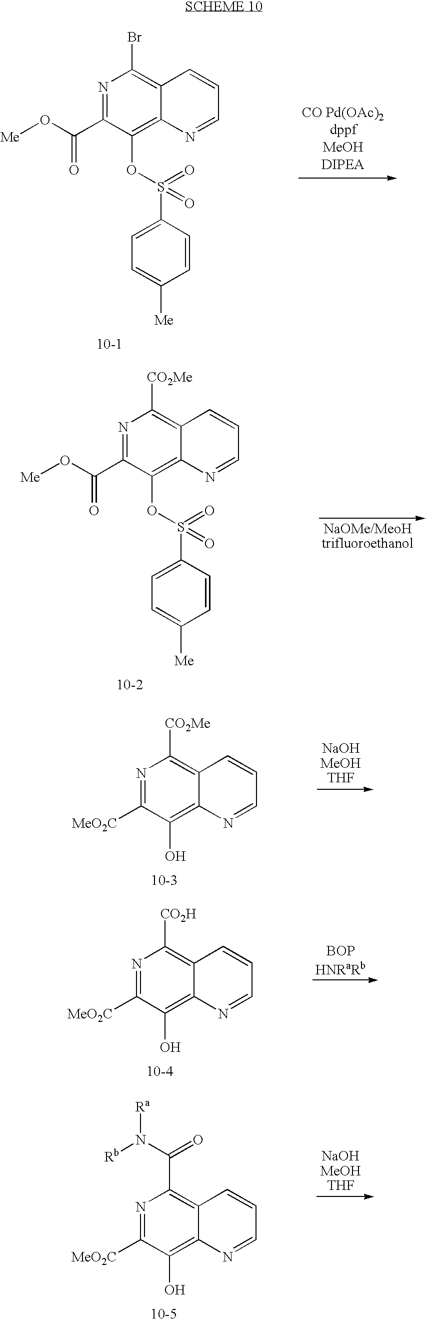

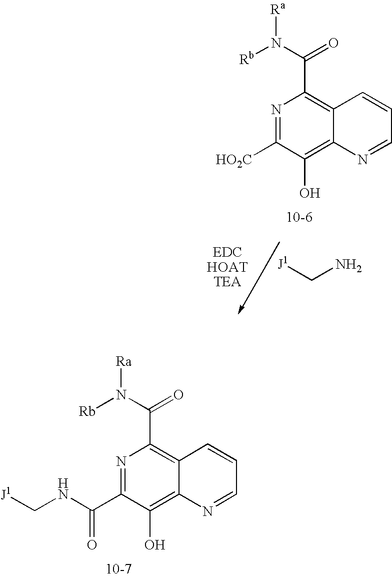

In the processes for preparing active drug substances for use in the pharmaceutical compositions of the present invention as set forth in the foregoing schemes, functional groups in various moieties and substituents may be sensitive or reactive under the reaction conditions employed and/or in the presence of the reagents employed. Such sensitivity/reactivity can interfere with the progress of the desired reaction to reduce the yield of the desired product, or possibly even preclude its formation. Accordingly, it may be necessary or desirable to protect sensitive or reactive groups on any of the molecules concerned. Protection can be achieved by means of conventional protecting groups, such as those described in Protective Groups in Organic Chemistry, ed. J. F. W. McOmie, Plenum Press, 1973 and in T. W. Greene & P. G. M. Wuts, Protective Groups in Organic Synthesis, John Wiley & Sons, 1991. The protecting groups may be removed at a convenient subsequent stage using methods known in the art. Alternatively the interfering group can be introduced into the molecule subsequent to the reaction step of concern. For example, if one or more of the substituents R¹, R² and R³ in amine 1-1 can interfere with the coupling reaction between reactants 1-1 and 1-2 of Scheme 1, the substituent can be incorporated into the molecule in a post-coupling step. Scheme 9 above illustrates the post-coupling introduction of an amide-containing substituent on the benzyl ring.

Further description of methods suitable for use (either directly or via routine modification) in preparing the 8-hydroxy-1,6-naphthyridine-7-carboxamide compounds employed in the present invention can be found in U.S. ______ which is published U.S. application Ser. No. 09/973,853, filed Oct. 10, 2001, the disclosure of which is incorporated herein by reference in its entirety. Further description of methods suitable for use (either directly or via routine modification) in preparing the 8-hydroxy-1,6-naphthyridine-7-carboxamide compounds employed in the present invention can also be found in WO 02/30930, the disclosure of which is incorporated herein by reference in its entirety.

The following examples serve only to illustrate the invention and its practice. The examples are not to be construed as limitations on the scope or spirit of the invention.

### EXAMPLE 1

### Preparation of 1,4-Butanesultam

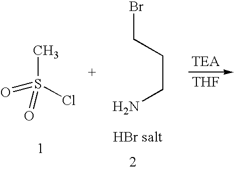

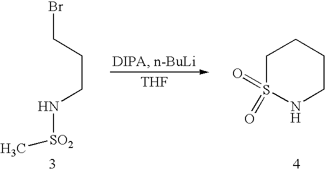

| | Weight | FW | Moles | Equiv. | Density | Volume |
|---|---|---|---|---|---|---|
| MsCl (1) | 2.36 Kg | 114.55 | 20.6 | 1.03 | 1.480 | 1.59 L |
| 3-bromo- | 4.40 Kg | 220 | 20.0 | 1.00 | | |
| propylamine | | | | | | |
| (2) HBr salt | | | | | | |
| TEA | 4.07 Kg | 101.19 | 40.2 | 2.01 | 0.726 | 5.60 L |
| THF | | | | | 43 + 4 + 8 = 55 L | |
| DIPA | 481 g | 101.19 | 4.75 | 0.25 | 0.722 | 666 mL |
| 1,10-Phenan- | 4.11 g | 180.21 | | | | |
| throline | | | | | | |
| n-BuLi, 1.6 M | | | | | | |
| in hexane | | | | | | |

The 3-bromopropylamine-HBr salt (2) and THF (43 L) were placed in a 72 L round-bottomed-flask under N₂ and the resulting slurry was cooled to 0° C. Two dropping funnels were fitted to the flask. One was charged with the TEA and the other with a solution of the MsCl (1) and THF (4 L). The contents of the addition funnels were added at roughly the same rate (the TEA was added slightly faster than the MsCl) while maintaining an internal reaction temperature below 10° C. The addition required 2 h. The resulting white suspension was warmed to 23° C. and aged for 1 h. The suspended solids (a mixture of TEA-HBr and TEA-HCl) were removed by filtration through a dry frit. The cake was washed with THF (8L). The combined filtrate and cake-rinse, a THF solution of 3, was collected in a 100 L round-bottomed-flask under N₂. To the solution of 3 was added the 1,10-phenanthroline and the DIPA and the resulting solution was cooled to −30° C. The n-BuLi was added over about 4 h maintaining the internal temperature below −20° C. After 1.25 eq of the n-BuLi was added the reaction mixture became deep brown and the color remained as the addition was completed. The reaction mixture was warmed to 0° C. over 3 h. A small aliquot was removed, and partitioned between saturated NH₄Cl and EtOAc. The EtOAc was evaporated and the residue examined by ¹H NMR to confirm consumption of 3 and conversion to 4. To the reaction mixture at 0° C. was added saturated aqueous NH₄Cl (12 L, the first 1 L slowly, a heat kick to 6° C. was observed) and then brine (12 L). The phases were partitioned and the aqueous phase was extracted with EtOAc (20 L). The organic phases were combined, washed with brine (4 L) and then concentrated under vacuum to about 12 L. The solvent was switched to EtOAc (20 L used) maintaining a volume of 12 L. After the solvent switch, a yellow slurry resulted. n-Heptane (20 L) was added with stirring and the slurry was cooled to 5° C. After a 1 h age the solids were collected on a frit and rinsed with cold (5° C.) 3:5 EtOAc/n-heptane. The wet cake was dried for 24 h under a stream of dry N₂ to provide 1.44 Kg (53% from 2) of sultam 4 as a crystalline yellow solid.

¹H NMR (CDCl₃, 400 mHz) δ 4.36 (br s, 1H), 3.45 (m, 2H), 3.10 (m, 2H), 2.24 (m, 2H), 1.64 (m, 2H).

### EXAMPLE 2

### Preparation of 5-(1,1-dioxido-1,2-thiazinan-2-yl)-N-(4-fluorobenzyl)-8-hydroxy-1,6-naphthyridine-7-carboxamide from methyl 5-bromo-8-hydroxy-1,6-naphthyridine-7-carboxylate

### Step 1: 5-Bromo-8-hydroxy-1,6-naphthyridine-7-carboxylic acid methyl ester

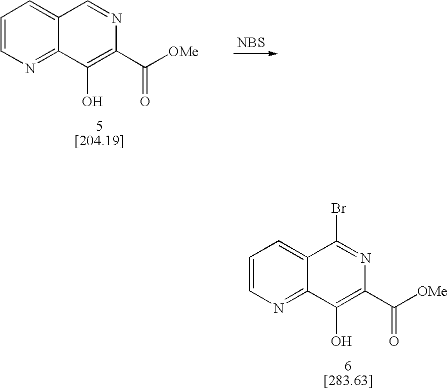

N-bromosuccinimide (7.83 g, 44.0 mmol) was added to a solution of 8-hydroxy-1,6-naphthyridine-7-carboxylic acid methyl ester (5, 8.17 g, 40.0 mmol) in chloroform (32 mL) over 20 min maintaining the temperature at 20-50° C. and the mixture was aged for 30 min at 50° C. The mixture became a thick, stirrable slurry and HPLC analysis indicated <2% starting material remaining. The mixture was cooled to 30° C. over 15 min. MeOH (64 mL) was added over 30 min then a 1:1 mixture of MeOH-water (64 mL) was added over 30 min. The mixture was cooled to −40° C. over 30 min and aged at −40° C. for 30 min. The cold mixture was filtered and the solid was washed with 1:1 MeOH:water (100 mL) at 10-20° C. The off white crystalline solid was dried under a stream of nitrogen to provide 10.48 g (93% yield) of 5-bromo-8-hydroxy-1,6-naphthyridine-7-carboxylic acid methyl ester (6).

HPLC retention times: 5=2.2 min, 6=6.0 min, HPLC conditions: 150×4.6 mm ACE 3 C18 column, isocratic elution with 30% MeCN in 0.025% aq H₃PO₄ at 1 mL/min, 25° C. with detection at 254 nm;

HPLC retention times: 5=1.8 min, 6=3.1 min, BPLC conditions: 150×4.6 mm ACE 3 C18 column, isocratic elution with 46% MeCN in 0.025% aq H₃PO₄ at 1 mL/min, 25° C. with detection at 254 nm.

¹³C NMR of 6 (CDCl₃, 100 MHz): 169.7, 156.3, 154.5, 143.9, 137.1, 132.4, 128.0, 126.1, 124.2, 53.4.

### Step 2: 5-Bromo-8-(4-toluenesulfonyloxy)-1,6-naphthyridien-7-carboxylic acid methyl ester

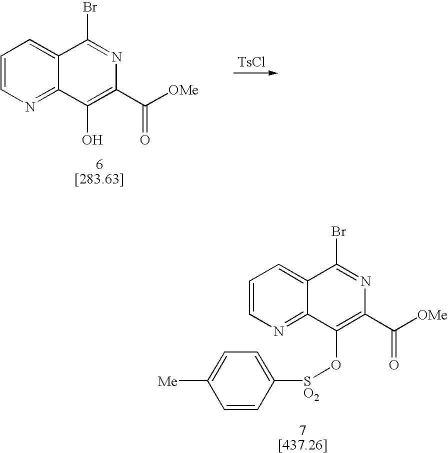

Triethylamine (0.759 g, 7.50 mmol) was added to a suspension of 5-bromo-8-hydroxy-1,6-naphthyridine-7-carboxylic acid methyl ester (6, 1.415 g, 5.000 mmol) in chloroform (5 mL) over 5 min maintaining the temperature at 20-50° C. to give a yellow suspension. p-Toluenesulfonyl chloride (1.15 g, 6.00 mmol) was added over 5 min maintaining the temperature at 20-40° C. to give a yellow solution. The mixture was aged at 40° C. for 2 h during which a crystalline solid precipitated out of the mixture and the color faded (HPLC analysis indicated <0.5% starting material remaining). The mixture was cooled to 20° C. over 15 min. MeOH (10 mL) was added over 30 min then a 1:1 mixture of MeOH:water (10 mL) was added over 30 min. The mixture was cooled to −40° C. over 30 min and aged at −40° C. for 30 min. The cold mixture was filtered and the solid was washed with 1:1 MeOH:water (10 mL), MeOH (5 mL), MTBE (10 mL) and hexanes (10 mL) all at 10-20° C. The off-white crystalline solid was dried under a stream of nitrogen to provide 2.112 g (97% yield) of 5-bromo-8-(p-toluenesulfonyloxy)-1,6-naphthyridine-7-carboxylic acid methyl ester (7).

HPLC retention times: 6=3.1 min, 7=12.4 min, HPLC conditions: 150×4.6 mm ACE 3 C18 column, isocratic elution with 46% MeCN in 0.025% aq H₃PO₄ at 1 mL/min, 25° C. with detection at 254 nm.

¹³C NMR of 7 (d6-DMSO, 100 MHz): 163.2, 157.0, 146.5, 145.8, 141.9, 141.3, 139.2, 137.2, 132.3, 130.4, 129.0, 127.6, 127.1, 53.3, 21.7.

### Step 3: 5-(1,1-Dioxido-1,2-thiazinan-2-yl)-8-(4-toluenesulfonyloxy)-1,6-naphthyridine-7-carboxylic acid methyl ester

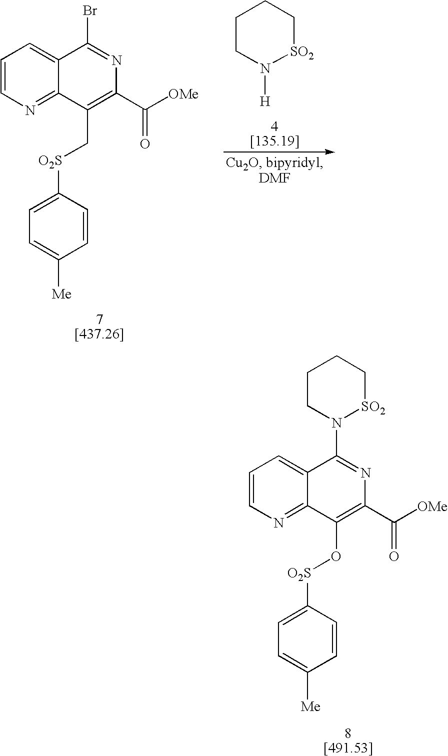

A mixture of 5-bromo-8-(p-toluenesulfonyloxy)-1,6-naphthyridine-7-carboxylic acid methyl ester (2, 2.186 g, 5.000 mmol), 1,4-butane sultam (4, 811 mg, 6.00 mmol), copper (I) oxide (858 mg, 6.00 mmol, <5 micron), 2,2′-bipyridyl (937 mg, 6.00 mmol) and DMF (10 mL) was degassed by stirring under a stream of nitrogen for 1 min and heated to 120° C. for 4 h. The brown suspension became a dark red solution with a small amount of undissolved copper (I) oxide remaining (HPLC analysis indicated <0.5% starting material remaining). The mixture was diluted with chloroform (10 mL), Solkaflok (200 mg) was added and the resulting mixture was filtered through a plug of Solkaflok. The plug was washed with chloroform (10 mL) and the combined filtrates were stirred vigorously with a solution of EDTA disodium salt dihydrate (3.8 g, 10.2 mmol) in water (40 mL) while air was slowly bubbled in for 40 min. The upper aqueous phase became turquoise while the lower organic phase became yellow. The organic phase was washed with a solution of EDTA disodium salt (1.9 g, 5.1 mmol) in water (30 mL) and a solution of sodium bisulfate monohydrate (0.87 g, 6.3 mmol) in water (30 mL). Each of the above three aqueous phases was back extracted sequentially with one portion of chloroform (15 mL). The organic phases were dried over sodium sulfate and filtered. The dried organic extracts were concentrated and solvent switched to a final volume of 15 mL MeOH using a total of 30 mL MeOH for the switch at atmospheric pressure. Product crystallized during the solvent switch. The resulting slurry was cooled to 0° C. over 30 min and aged at 0° C. for 30 min. The slurry was filtered cold and the solid was washed with MeOH (15 mL). The off white solid was dried under a stream of nitrogen to provide 1.910 g (78%) of 5-(N-1,4-butanesultam)-8-(p-toluenesulfonyloxy)-1,6-naphthyridine-7-carboxylic acid methyl ester (8).

HPLC retention times: 7=12.4 min, 8=10.3 min, DMF=1.3 min, Bipy=1.5 min, HPLC conditions: 150×4.6 mm ACE 3 C18 column, isocratic elution with 46% MeCN in 0.025% aq H₃PO₄ at 1 mL/min, 25° C. with detection at 254 nm.
¹³C NMR of 8 (CDCl₃, 100 MHz): 164.2, 155.3, 151.9, 146.7, 145.4, 141.2, 137.8, 135.3, 133.6, 129.6, 128.9, 125.4, 124.3, 53.4, 52.9, 48.7, 24.2, 22.0, 21.7.

### Step 4: 5-(1,1-Dioxido-1,2-thiazinan-2-yl)-8-hydroxy-1,6-naphthyridine-7-carboxylic acid methyl ester

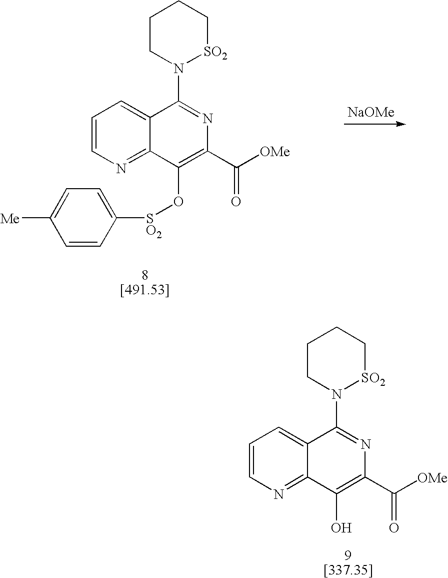

5-(N-1,4-butanesultam)-8-(p-toluenesulfonyloxy)-1,6-naphthyridine-7-carboxylic acid methyl ester (8, 1.597 g, 3.250 mmol) was dissolved in DMF (3.25 mL) at 40° C. and transferred to a solution of 0.5M NaOMe in MeOH (16.25 mL, 8.125 mmol) over ca 1-2 min at 20-25° C. The resulting yellow homogenous mixture was heated to 50° C. and aged for 5 min (HPLC analysis indicated <0.5% starting material remaining). Mixture was cooled to 25° C. over 15 min and aged at 25° C. for 15 min during which a yellow crystalline precipitate was deposited. Acetic acid (390 mg, 6.50 mmol) was added over 1 min (yellow color faded) then water (32.5 mL) was added over 15 min at 25° C. The slurry was aged for 30 min 25° C. and filtered. The filter cake was washed with 1:1 MeOH:water (32.5 mL) and then with 1:1 MTBE:hexanes (8 mL). The filter cake was dried under a stream of nitrogen to provide 1.064 g (97%) of 5-(N-1,4-butanesultam)-8-hydroxy-1,6-naphthyridine-7-carboxylic acid methyl ester (9) as an off white crystalline solid.

HPLC retention times: 8=10.3 min, 9=2.9 min, HPLC conditions: 150×4.6 mm ACE 3 C18 column, isocratic elution with 46% MeCN in 0.025% aq H₃PO₄ at 1 mL/min, 25° C. with detection at 254 nm.
¹³C NMR of 9 (d6-DMSO, 100 MHz): 167.8, 154.4, 153.5, 143.9, 143.7, 135.2, 125.9, 125.2, 124.4, 53.2, 53.1, 49.1, 24.4, 21.9.

### Step 5: 5-(1,1-Dioxido-1,2-thiazinan-2-yl)-N-(4-fluorobenzyl)-8-hydroxy-1,6-naphthyridine-7-carboxamide (Compound A), monoethanolate

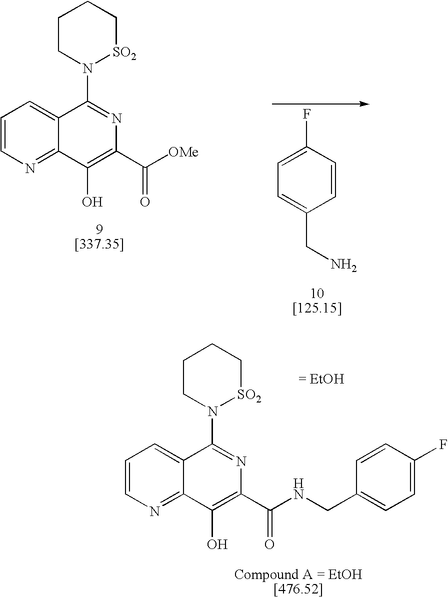

A suspension of 5-(N-1,4-butanesultam)-8-hydroxy-1,6-naphthyridine-7-carboxylic acid methyl ester (9, 1.012 g, 3.00 mmol) and 4-fluorobenzylamine (10, 1.314 g, 10.5 mmol) in EtOH (9.0 mL) was heated to 75-77° C. for 2 h during which the mixture became a yellow homogeneous solution (HPLC analysis indicated <0.5% starting material remaining). Acetic acid (0.630 mg, 10.5 mmol) was added over 1 min (yellow color faded) then water (9.0 mL) was added over 10 min at 75° C. An off white crystalline solid began to precipitate near the end of addition of the water. The slurry was cooled to 0° C. over 30 min then aged for 30 min at 0° C. and filtered. The filter cake was washed with 5% HOAc in 1:1 EtOH:water (5 mL) then with 1:1 EtOH:water (10 mL) and then with EtOH (5 mL). The filter cake was dried under a stream of nitrogen to provide 1.343 g (94%) of the monoethanolate of 5-(N-1,4-butanesultam)-N-(4-fluorobenzyl)-8-hydroxy-1,6-naphthyridine-7-carboxamide (Compound A) as an off white crystalline solid.

HPLC retention times: 9=2.9 min, Compound A=6.7 min, 10=1.4 min, impurity present in 10=4.3 min, HPLC conditions: 150×4.6 mm ACE 3 C18 column, isocratic elution with 46% MeCN in 0.025% aq H₃PO₄ at 1 mL/min, 25° C. with detection at 254 nm;

HPLC retention time: 9=10.9 min, HPLC conditions: 150×4.6 mm ACE 3 C18 column, isocratic elution with 24% MeCN in 0.025% aq H₃PO₄ at 1 mL/min, 25° C. with detection at 254 nm.

¹H NMR (d6-DMSO, 400 MHz): 9.25 (t, J=6.4, 1H), 9.16 (d, J=8.4, 1H), 8.56 (d, J=8.4, 1H), 7.86 (dd, J=8.4, 4.1, 1H), 7.41 (dd, J=8.4, 5.7, 2H), 7.16, t, J=8.8, 2H), 4.60 (d, 6.3, 2H), 4.00-3.70 (m, 2H), 3.65-3.45 (m, 2H), 2.35-2.10 (m, 3H), 1.7 (m, 1H).

### Step 6: Sodium salt of 5-(1,1-Dioxido-1,2-thiazinan-2-yl)—N-(4-fluorobenzyl)-8-hydroxy-1,6-naphthyridine-7-carboxamide

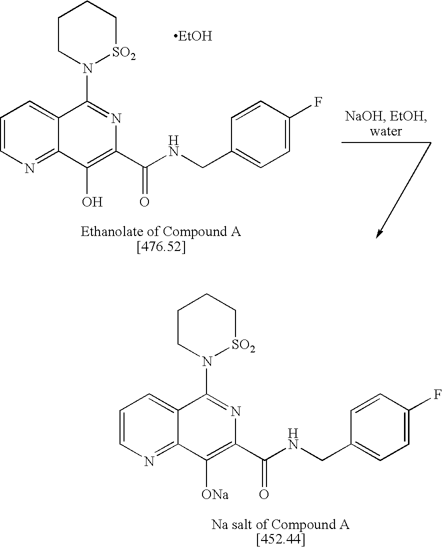

5-(N-1,4-Butanesultam)-N-(4-fluorobenzyl)-8-hydroxy-1,6-naphthyridine-7-carboxamide (Compound A) monoethanolate (1.207 g, 2.533 mmol) was dissolved in a mixture of EtOH (24 mL) and water (11 mL) by heating to 78° C. for 1 h. A solution of 5M aq NaOH (0.608 mL, 3.04 mmol) was added over 15 min at 78° C. A yellow crystalline precipitate was deposited. The mixture was aged at 78° C. for 20 min, then cooled to 20° C. over 30 min and aged for 30 min at 20° C. The slurry was filtered and the filter cake was washed with 2:1 EtOH:water (5 mL) and EtOH (15 mL). The filter cake was dried under a stream of nitrogen to provide 1.088 g (95%) of 5-(N-1,4-butanesultam)-N-(4-fluorobenzyl)-8-hydroxy-1,6-naphthyridine-7-carboxamide sodium salt (Compound A sodium salt) as a yellow crystalline solid.

The Na salt was analyzed by differential scanning calorimetry at a heating rate of 10° C./min in an open cup under flowing nitrogen and was found to have a DSC curve exhibiting an endotherm with a peak temperature of about 348° C. and an associated heat of fusion of about 45 J/gm followed by an exotherm with a peak temperature of about 352° C. and an associated heat of fusion of about 45 J/gm.

The XRPD pattern of the Na salt was generated on a Philips Analytical X-ray powder diffraction instrument with XRG 3100 generator using a continuous scan from 2 to 40 degrees 2 theta over about 126 minutes. The resulting XRPD pattern was analyzed using Philips X'Pert Graphics and Identify software. Copper K-Alpha 1 radiation was used as the source. The experiment was run under ambient conditions. The XRPD pattern was found to have characteristic diffraction peaks corresponding to d-spacings of 12.63, 5.94, 5.05, 4.94, 4.81, 4.61, 4.54, 4.34, 3.88, 3.73, 3.49, 3.45, 3.22, 3.15, 3.12, and 2.86 angstroms.

### EXAMPLE 3

### Sodium 5-(1,1-dioxido-1,2-thiazinan-2-yl)-7-[({4-fluoro-2-[(methylamino)carbonyl]-benzyl}amino)carbonyl]-1,6-naphthyridin-8-olate

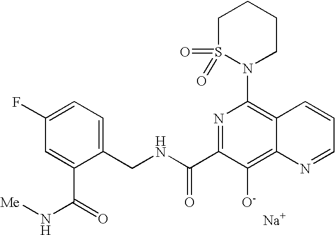

### Step 1: 1-(Bromomethyl)-4-fluoro-2-iodobenzene

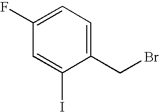

A suspension of 4-fluoro-2-iodotoluene (14.3 g, 60.6 mmol, Lancaster Synthesis), N-bromosuccinimide (16.2 g, 90.9 mmol), and benzoyl peroxide (0.74 g, 3.0 mmol) in carbon tetrachloride (500 mL) was heated to reflux for 3 days. Additional NBS (0.5 eq portions) was added as needed over this period to drive the reaction to completion. The reaction was cooled, filtered, and the filtrate was concentrated in vacuo. The residue was purified by flash column chromatography (ISCO column, 110 g silica gel) eluting with 100% hexane to afford the desired product as a white solid.

¹H NMR (DMSO-d6, 400 MHz) δ 7.79 (1H, dt, J=8.4, 1.3 Hz), 7.68 (1H, m), 7.31 (1H, m), and 4.74 (2H, s) ppm.

### Step 2: 1-(Azidomethyl)-4-fluoro-2-iodobenzene

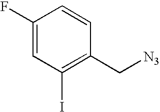

A suspension of 1-(bromomethyl)-4-fluoro-2-iodobenzene (15.68 g, 47.8 mmol) and sodium azide (4.21 g, 64.7 mmol) in dry DMF (30 mL) was heated to 50° C. for six hours. The reaction was filtered and the filtrate was concentrated in vacuo to a volume of about 10 mL. The crude was purified by flash column chromatography (ISCO column, 110 g silica gel) eluting with 100% hexane to afford the desired product as a clear oil.

¹H NMR (DMSO-d6, 400 MHz) δ 7.83 (1H, dd, J=8.3, 2.7 Hz), 7.54 (1H, dd, J=8.6, 6.1 Hz), 7.33 (1H, td, J=8.5, 2.6 Hz), and 4.52 (2H, s) ppm.

### Step 3: 1-(4-Fluoro-2-iodophenyl)methanamine

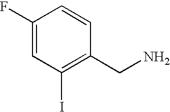

Triphenylphosphine (13.2 g, 50.4 mmol) was added to 1-(azidomethyl)-4-fluoro-2-iodobenzene (9.31 g, 33.6 mmol) dissolved in dry DMF (20 mL) at 0° C. After one hour water (3.03 mL, 168 mmol) was added and the solution was heated to 55° C. for one hour. The reaction was cooled and the solution was concentrated to about 10 mL in vacuo. The residue was purified in two runs by preparative HPLC (Gilson semi preparative HPLC system using a Waters Delta pak column (3(10×40 mm I.D.) cartridges, C18, 15 μM pore size) eluting with 95-5% water (0.025% TFA)/acetonitrile (0.025% TFA) at 45 mL/min) to give the desired product in 75% purity. Using a Waters OASIS MCX Cartridge (6 g, 35 cc syringe), half of the 75% pure product dissolved in methanol was loaded onto the column pre-equilibrated with a 1:1 solution of water and methanol. The column was washed once with the 1:1 solution and then washed several times with methanol to remove all UV active material. The amine was eluted by washing the column with methanol saturated with ammonia gas. This procedure was repeated on the remaining 75% pure product. The two batches were combined and concentrated in vacuo to give the free base of the desired product as a yellow oil.

¹H NMR (DMSO-d6, 400 MHz) δ 8.28 (2H, bs), 7.86 (1H, dd, J=8.2, 2.7 Hz), 7.53 (1H, dd, J=8.6, 5.9 Hz), 7.41 (1H, td, J=8.5, 2.6 Hz), and 4.09 (2H, s) ppm.

### Step 4: Tert-butyl 4-fluoro-2-iodobenzylcarbamate

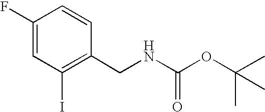

Triethylamine (1.41 mL, 10.1 mmol) was added to a 0° C. suspension of 1-(4-fluoro-2-iodophenyl)methanamine (2.30 g, 9.16 mmol) and di-tert-butyl dicarbonate (2.20 g, 10.1 mmol) in dry methylene chloride (60 mL). The homogeneous solution was stirred at 0° C. for five minutes then at room temperature for two hours. The reaction was diluted with methylene chloride (30 mL), washed with water three times and washed once with brine. The organic layer was dried over sodium sulfate, filtered, and concentrated in vacuo to a clear oil. The residue was purified by flash column chromatography (ISCO column, 110 g silica gel) eluting with a 10-25% ethyl acetate/hexane gradient over 30 minutes to afford the desired product as a clear oil.

¹H NMR (CDCl₃, 400 MHz) δ 7.55 (1H, dd, J=8.0, 2.5 Hz), 7.34 (1H, t, J=7.1 Hz), 7.05 (1H, td, J=8.3, 2.4 Hz), 5.02 (1H, m) 4.31 (2H, d, J=6.0 Hz) and 1.46 (9H, s) ppm.

ES HRMS: calc'd for C₁₂H₁₅FINO₂+Na 374.0024, observed 374.0022.

### Step 5: Tert-butyl 4-fluoro-2-[(methylamino)carbonyl]benzylcarbamate

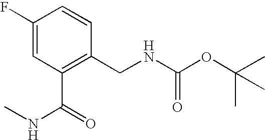

Through a solution of tert-butyl 4-fluoro-2-iodobenzylcarbamate (1.00 g, 2.85 mmol) in dry DMF (20 mL), in an oven dried glass insert in a high pressure bomb reactor flushed with nitrogen, was bubbled methylamine gas at 0° C. until the solution was saturated. Diisopropylethylamine (0.99 mL, 5.70 mmol), palladium acetate (64 mg, 0.29 mmol) and 1,1′-bis(diphenylphosphino)ferrocene (158 mg, 0.29 mmol) were added to the saturated solution. The glass insert was then placed in the pressure vessel and the vessel was purged once with carbon monoxide gas. The vessel was recharged with carbon monoxide gas to pressure of 300 psi, placed into an oil bath, and heated to 90° C. for four hours. The vessel was cooled, the gas was released slowly and the resulting mixture was partitioned between water and ethyl acetate. The layers were separated and the organic extracts were dried over sodium sulfate, filtered, and concentrated in vacuo to a brown liquid. The residue was purified by flash column chromatography (ISCO column, 110 g silica gel) eluting with a 10-50% acetone/hexane gradient over 35 minutes to afford the desired product as a brown crystalline solid.

¹H NMR (DMSO-d6, 400 MHz) δ 8.38 (1H, d, J=4.0 Hz), 7.35−7.19 (4H, m), 4.20 (2H, d, J=6.1 Hz), 2.75 (3H, d, J=4.6 Hz) and 1.39 (9H, s) ppm.
Step 6: {4-Fluoro-2-[(methylamino)carbonyl]phenyl}methanaminium chloride

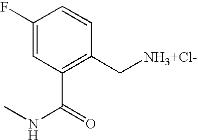

Hydrogen chloride gas was bubbled through a −78° C. solution of tert-butyl 4-fluoro-2-[(methylamino)carbonyl]benzylcarbamate (615 mg, 2.18 mmol) in ethyl acetate (20 mL) until the solution was saturated. The flask was then allowed to warm to room temperature. The reaction was concentrated in vacuo to a volume of about 5 mL and the flask was capped and placed in the freezer overnight. In the morning, the solids that had precipitated were collected by vacuum filtration and washed with cold ethyl acetate to give the desired product as an off-white solid.

¹H NMR (DMSO-d6, 400 MHz) δ 8.81 (1H, d, J=4.0 Hz), 8.25 (3H, bs), 7.62 (1H, dd, J=8.3, 5.7 Hz), 7.50−7.42 (2H, m), 4.04 (2H, s), and 2.80 (3H, d, J=3.7 Hz) ppm.

### Step 7: 5-(1,1-Dioxido-1,2-thiazinan-2-yl)-8-hydroxy-1,6-naphthyridine-7-carboxylic acid

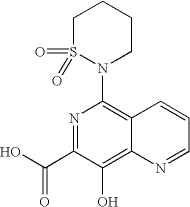

A suspension of methyl 5-(1,1-dioxido-1,2-thiazinan-2-yl)-8-hydroxy-1,6-naphthyridine-7-carboxylate (1.00 g, 2.96 mmol, prepared as described in Example 2 above) in methanol (18 mL) with aqueous lithium hydroxide (17.8 mL, 17.8 mmol, 1N solution) was stirred overnight at 60° C. The suspension was acidified to a pH=4 using 3N HCl (about 6 mL) and the resulting solution was allowed to stir overnight at room temperature. In the morning, the solids that had precipitated out of solution were collected by vacuum filtration to give the desired product as a light yellow solid.

¹H NMR (DMSO-d6, 400 MHz) δ 9.21 (1H, dd, J=4.3, 1.6 Hz), 8.62 (1H, dd, J=8.5, 1.6 Hz), 7.92 (1H, dd, J=8.5, 4.3 Hz), 3.91−3.78 (2H, m), 3.55−3.45 (2H, m), 2.28 (3H, m) and 1.64 (1H, m) ppm.

### Step 8: 5-(1,1-Dioxido-1,2-thiazinan-2-yl)-N-{4-fluoro-2-8 (methylamino)carbonyl]benzyl}-8-hydroxy-1,6-naphthyridine-7-carboxamide (Compound B)

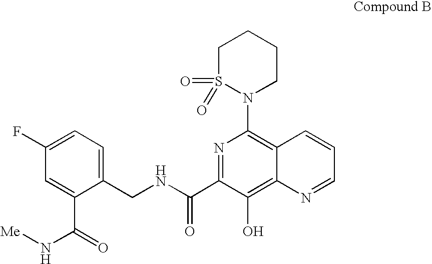

A solution of 5-(1,1-dioxido-1,2-thiazinan-2-yl)-8-hydroxy-1,6-naphthyridine-7-carboxylic acid (100 mg, 0.31 mmol), 1-[3-(dimethylamino)propyl]-3-ethylcarbodiimide hydrochloride (89 mg, 0.46 mmol), 1-hydroxy-7-azabenzotriazole (63 mg, 0.46 mmol), {4-fluoro-2-[(methylamino)carbonyl]phenyl}-methanaminium chloride (101 mg, 0.46 mmol) and triethylamine (65 μL, 0.46 mmol) in dry DMF (2 mL) was stirred at room temperature overnight. In the morning, a couple drops of water were added and the reaction was filtered through a glass fiber filter. The filtrated was purified by preparative HPLC (Gilson semi preparative HPLC system using a Waters Nova pak column (10×40 mm I.D. cartridges, C18, 6 μM pore size) eluting with 95-5% water (0.025% TFA)/acetonitrile (0.025% TFA) at 35 mL/min) to give the product as a TFA salt. The crude solid was dissolved in CHCl₃ and washed with aqueous saturated ammonium chloride solution. The aqueous layer was back-extracted with CHCl₃ three times and the combined organic extracts were dried over sodium sulfate, filtered, and concentrated in vacuo to give the desired product as a pale yellow solid.

¹H NMR (DMSO-d6, 400 MHz) δ 9.53 (1H, s), 9.19 (1H, s), 8.68 (1H, s), 8.58 (1H, d, J=8.0 Hz), 7.89 (1H, d, J=3.8 Hz), 7.53 (1H, m), 7.41−7.34 (2H, m), 4.64 (2H, d, J=5.7 Hz), 3.92−3.47 (4H, m), 2.83 (3H, d, J=3.8 Hz), 2.35 (3H, m), and 1.64 (1H, m) ppm.

### Step 9: Sodium 5-(1,1-dioxido-1,2-thiazinan-2-yl)-7-[({4-fluoro-2-[(methylamino)carbonyl]benzyl}amino)carbonyl]-1,6-naphthyridin-8-olate

Sodium hydroxide (150 μL, 0.15 mmol, 1N solution) was added to a cloudy solution of 5-(1,1-dioxido-1,2-thiazinan-2-yl)-N-{4-fluoro-2-[(methylamino)carbonyl]benzyl}-8-hydroxy-1,6-naphthyridine-7-carboxamide (73 mg, 0.15 mmol) in a 2 mL mixture of acetone, acetonitrile and water. The homogeneous bright yellow solution was allowed to stir at room temperature for 30 minutes. The solvent was removed in vacuo and dried overnight on the high vac with gentle heating to give the desired product as a bright yellow solid.

¹H NMR (DMSO-d6, 400 MHz) δ 12.12 (1H, s), 8.78 (1H, m), 8.66 (1H, d, J=4.6 Hz), 8.29 (1H, d, J=6.8 Hz), 7.56 (1H, dd, J=8.4, 4.2 Hz), 7.46 (1H, dd, J=8.3, 5.6 Hz), 7.26−7.19 (2H, m), 4.61 (2H, d, J=5.9 Hz), 3.81 (2H, m), 3.51 (1H, m), 3.23 (1H, m), 2.81 (3H, d, J=4.4 Hz), 2.43 (1H, m), 2.23 (2H, m) and 1.64 (1H, m) ppm.

ES HRMS: calc'd for C₂₂H₂₁FN₅NaO₅S 510.1218, observed 510.1219.

### EXAMPLE 4

Wet-Granulated Compressed Tablets of Compound A

| | Amount per | Amt per batch |
|---|---|---|
| Ingredient | Tablet (mg) | (wt. percent) |
| Compound A Na salt¹ | 106 | 26.5 |
| (on free phenol basis) | (100) | (25.2) |
| lactose | 183.2 | 45.8 |
| poloxamer 407 | 3.6 | 0.9 |
| hydroxypropyl cellulose | 9.6 | 2.4 |
| croscarmellose sodium | 9.6 | 2.4 |
| Avicel² (EG)³ | 80.0 | 20.0 |
| magnesium stearate (EG)³ | 8.0 | 2.0 |

¹Crystalline monosodium salt of Compound A (see Example 2, Step 6) milled to a mean particle size of about 6 μm.
²Avicel = microcrystalline cellulose.
³EG = extragranular.

The Compound A sodium salt (6.4 g on a free phenol basis), lactose, croscarmellose sodium and hydroxypropyl cellulose were blended together in a high shear granulator for 2 minutes, after which a 5.13 wt. % solution of the poloxamer 407 in purified USP water in an amount equivalent to 25% (w/w) of the dry powder mix was added to the blend in the high shear granulator over a period of about 3 minutes to form a wet granulate. The granulated mixture was dried in an oven at 40° C. for a period of at least 12 hours. The dried granules were de-lumped by passing through a #30 mesh sieve. The de-lumped granules were then blended in a Turbula mixer (Willy A. Bachofen A G, Basel, Switzerland) with the microcrystalline celluose for about 4 minutes, and the blended mixture was then lubricated with the magnesium stearate in the Turbula mixer for about 3 minutes. The lubricated mixture was manually compressed into 13/32 inch image tablets using a Carver press (Fred S. Carver Inc., Menomonee Falls, Wis.) at 1400 kgf (1.4 ton).

### EXAMPLES 5-7

Wet-Granulated Compressed Tablets of Compound A

Compressed tablets containing 100 mg of Compound A on a free phenol basis and having the following compositions were prepared:

| | Example 5 | Example 6 | Example 7 |
|---|---|---|---|
| Ingredient | (wt. %) | (wt. %) | (wt. %)) |
| Compound A Na salt¹ | 26.2 | 26.1 | 26.2 |
| (on free phenol basis) | (25.0) | (24.9) | (25.0) |
| lactose | 46.0 | 44.0 | 47.0 |
| Tween 80² | 1.0 | — | — |
| sodium lauryl sulfate | — | 1.5 | — |
| hydroxypropyl cellulose | 2.4 | 2.4 | 2.4 |
| croscarmellose sodium | 2.4 | 4.0 | 2.4 |
| Avicel² (EG)³ | 20.0 | 20.0 | 20.0 |
| magnesium stearate (EG)³ | 2.0 | 2.0 | 2.0 |

¹Crystalline monosodium salt of Compound A (see Example 2, Step 6) milled to a mean particle size of about 3 μm (Examples 6 and 7) and 6 μm (Example 5).
²Tween 80 = polysorbate 80; Avicel = microcrystalline cellulose.
³EG = extragranular.

The tablets were prepared using a procedure substantially the same as that described in Example 4, except that in the procedure for Example 5 the microcrystalline cellulose and magnesium stearate were added to and blended with the granules in the Turbula mixer in a single step.

### EXAMPLES 8 & 9

Directly Compressed Tablets of Compound A

Tablets containing 100 mg of Compound A on a free phenol basis and having the following compositions have been prepared by direct compression:

| | Example 8 | Example 9 |
|---|---|---|
| Ingredient | (wt. %) | (wt. %)) |
| Compound A Na salt¹ | 27.3 | 26.2 |
| (on free phenol basis) | (26.0) | (25.0) |
| lactose | 64 | 69.3 |
| poloxamer 338 | 2.3 | — |
| hydroxypropyl cellulose | 2.2 | — |
| croscarmellose sodium | 2.9 | 3.0 |
| magnesium stearate (EG)² | 1.5 | 1.5 |

¹Crystalline monosodium salt of Compound A (see Example 2, Step 6) milled to a mean particle size of about 3 μm in Example 8 and 6 μm in Example 9.
²EG = extragranular

### PREPARATION OF EXAMPLE 8

A 5 wt. % aqueous suspension of the Compound A sodium salt (1.25 g on a free phenol basis) was prepared by mixing the salt, poloxamer 338 (0.5 wt. %), and hydroxypropyl cellulose (0.5 wt. %) in purified USP water. The suspension was lyophilized by freezing at −70° C. and drying overnight at −5° C. under vacuum. The dry powder was passed through a 30 mesh screen to delump, and then blended with the croscarmellose sodium, lactose and magnesium stearate using a Turbula mixer for 5 minutes. The lubricated blend was then manually compressed into tablets using a Carver press at 1400 kgf (1.4 ton).

### PREPARATION OF EXAMPLE 9

Example 9 was prepared in the same manner as Example 8 except that the poloxamer 338 and hydroxypropyl cellulose were not included.

### EXAMPLE 10

Film Coated Compressed Tablets Containing Compound A

| | Amount per | Amt per batch |
|---|---|---|
| Ingredient | Tablet (mg) | (wt. percent) |
| Tablet Core: | | |
| Compound A Na salt¹ | 105 | 26.25 |
| (on free phenol basis) | (100) | (25.0) |
| lactose | 184.0 | 46.0 |
| poloxamer 407 | 3.6 | 0.9 |
| hydroxypropyl cellulose | 9.6 | 2.4 |
| croscarmellose sodium | 9.6 | 2.4 |
| BHA | 0.20 | 0.05 |
| Avicel² (EG)³ | 80.0 | 20.0 |
| magnesium stearate (EG)³ | 8.0 | 2.0 |
| Film Coating: | | |
| Opadry I⁴ | 8.0 | 2.0 |

¹Crystalline monosodium salt of Compound A (see Example 2, Step 6) milled to a mean particle size of about 6 μm.
²Avicel = microcrystalline cellulose.
³EG = extragranular.
⁴The weight percent of Opadry I film coating is expressed as the percentage of the weight of the uncoated core tablet.

Uncoated compressed tablets containing 100 mg of Compound A on a free phenol basis were prepared in accordance with the procedure described in Example 4, except that the aqueous poloxamer solution also contained BHA. The tablets were then film coated with an aqueous coating suspension containing Opadry I (10 wt. %) in a pan coater to a coat weight of about 2 wt. % per tablet, wherein the coat is the dried form of the suspension.

### EXAMPLE 11

Pharmacokinetic Experiments in Dogs

Pharmacokinetic (PK) values for Compound A were determined in Beagle dogs orally dosed with compressed tablets prepared as described in Examples 4 to 9. Male, purpose-bred beagle dogs (Marshall Farms) were used in all the studies. The dogs were housed in an AAALAC-accredited facility in accordance with USDA guidelines. Studies were conducted under a protocol approved by the WP-IACUC. Dog weights were measured and recorded prior to dosing. Dog weights ranged from approximately 8 to 10 kg. Dogs having similar weights were employed in each of the studies. Three dogs were employed in each study, except that the study with tablets of Example 6 used four dogs. The dose was approximately 10 mg per kg of body weight (i.e., 10 mpk) in the studies using tablets of Examples 6 and 9, and was approximately 12 mpk in the studies using tablets of Examples 4, 5, 7 and 8.

Dosing: After an overnight fast, the dogs received tablet formulations dosed orally followed by 50 mL of water. Water was returned at 2 hours after dosing and food was returned 4 hours after dosing. Blood was drawn from 21 g catheters placed in the cephalic vein at pre-dose, and 0.25, 0.5, 1, 1.5, 2, 3, 4, 6, 8 and 24 hours after dosing. The plasma was separated by centrifugation (15 minutes at 2500 g) and stored overnight at −70° C. for LC/MS/MS the following day.

Sample preparation and analysis: The plasma samples from the dogs were extracted using automated (Tecan automated liquid handler) solid phase extraction. Samples were loaded on a Waters Oasis HLB 30 mg extraction cartridge. The cartridges were rinsed with 40% methanol:water and the drug was eluted with HPLC mobile phase. Plasma extracts were injected on a Phenomenex C18(2) column (4.6×50 mm). The sample extracts were ionized using an APCI interface and samples were monitored by selected reaction monitoring (SRM) in the positive ionization mode. The dynamic range of the LC/MS/MS assay was 5-10,000 ng/mL based on a 100 μL aliquot of dog plasma.

PK Calculations: AUC^{0-24 hrs} (area under the curve of plasma concentration of Compound A versus time) was calculated using the trapezoidal rule, as described in Shargel et al., Applied Biopharmaceutics & Pharmacokinetics, 4^{th} edition, 1999, pp. 8-9. Mean and standard deviations were calculated with Excel® 97 SR-2(f).

Results: Wet granulated compressed tablets containing a nonionic surfactant (i.e., Example 4 containing poloxamer 407 and Example 5 containing Tween 80) exhibited at least a three-fold increase in AUC relative to compressed tablets containing an anionic surfactant (Example 6 containing SLS) or no surfactant at all (Example 7).

This PK data is consistent with the results of in vitro experiments characterizing the ability of these surfactants to disperse Compound A. In these experiments, Compound A Na salt (milled to a 3 μm mean particle size) was suspended in acidified aqueous solutions of the surfactant, and the surfactants were ranked for effectiveness in dispersing Compound A based on a visual evaluation of the suspension for turbidity and particle size. The visual evaluations were confirmed by measuring the particle size distributions in the suspensions using a laser scattering particle size distribution analyzer. The results of these tests have shown that Compound A drug particles flocculate in aqueous media at physiological pHs in the absence of a nonionic surfactant or in the presence of SLS.

Directly compressed tablets containing a nonionic surfactant (i.e., Example 8 containing poloxamer 338) provided a 60% improvement in AUC as compared to a directly compressed tablet that contained no surfactant (Example 9).

### EXAMPLE 12

Dry Granule Filled Capsules of Compound A

| | Amount per |
|---|---|
| | batch |
| Ingredient | (wt. %) |
| Compound A Na salt¹ | 52.50 |
| (on free phenol basis) | (50.0) |
| lactose (monohydrate) | 12.23 |
| Avicel² | 12.23 |
| poloxamer 407 | 10.0 |
| hydroxypropyl cellulose-EXF | 2.0 |
| hydroxypropyl cellulose-SL | 3.0 |
| croscarmellose sodium | 4.0 |
| sodium stearyl fumarate (EG³) | 4.0 |
| BHA | 0.05 |
| Size 0 HPMC capsule shells⁴ | N/A |

¹crystalline monosodium salt of Compound A (see Example 2, Step 6) milled to a mean particle size of about 4 μm.
²Avicel = microcrystalline cellulose.
³EG = extragranular.
⁴HPMC = hydroxypropyl methylcellulose.

Dry granule filled capsules containing 200 mg of Compound A on a free phenol basis were prepared as follows: All of the ingredients listed above except for sodium stearyl fumarate and BHA were dry mixed in a 10 L Fielder mixer for two minutes and then wet granulated in the same mixer with an alcohol-water solution containing 95 wt. % ethanol (SD-3A) and 5% wt. % USP water. The alcohol-water solution was charged to the mixer in two portions. The first portion of the granulating fluid, amounting to 10 wt. % of the dry mix, contained the BHA. The second portion of the granulating fluid contained no BHA and brought the total granulating fluid level up to approximately 24 wt. % of the dry mix. The “wet” granules were subsequently past through a cone mill with a 375Q screen operating at 2000 RPM, then dried in a GPC-G3 fluid bed drier with the process air temperature set initially at 25° C. for 24 minutes. and then raised to 50° C. for additional 43 minutes. The granule LOD (loss on drying) after drying was 1.3 wt. %. The dry granules were then past through a cone milling with 50 G screen operating at 2000 RPM. The milled granules were then blended with EG sodium stearyl fumarate in a V-shell tumble blender for 5 minutes The blend was encapsulated using an H&K encapsulator with a 14.5 mm size 0 dosing disk to give the dry granule filled capsules.

When administered to humans, the poloxamer 407-containing capsules described above have exhibited AUCs comparable to that of the tablets described in Example 10 and approximately 50% higher than that of capsules having a similar composition but formulated without poloxamer 407.

While the foregoing specification teaches the principles of the present invention, with examples provided for the purpose of illustration, the practice of the invention encompasses all of the usual variations, adaptations and/or modifications that come within the scope of the following claims.

1. A pharmaceutical composition comprising a therapeutically effective amount of a compound of Formula (I), or a pharmaceutically acceptable salt thereof: 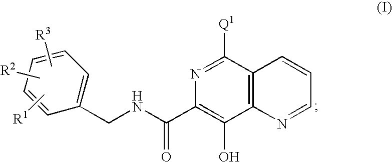
and a nonionic surfactant; wherein in Formula (I) each of R¹, R² and R³ is independently:
(1) —H,
(2) —C₁₋₆ alkyl, optionally substituted with one substituent which is —OH, —O—C₁₋₆ alkyl, —O—C₁₋₆ haloalkyl, —CN, —NO₂, —N(R^{a}R^{b}), —C(═O)N(R^{a}R^{b}), —C(═O)R^{a}, —CO₂R^{c}, —OCO₂R^{c}, —S(O)ₙR^{c}, —SO₂N(R^{a}R^{b}), —N(R^{a})C(═O)R^{b}, —N(R^{a})CO₂R^{c}, —N(R^{a})SO₂R^{c}, —N(R^{a})SO₂N(R^{a}R^{b}), —OC(═O)N(R^{a}R^{b}), or —N(R^{a})C(═O)N(R^{a}R^{b}),
(3) —O—C₁₋₆ alkyl, optionally substituted with one substituent which is —OH, —O—C₁₋₆ alkyl, —O—C₁₋₆ haloalkyl, —S(O)ₙR^{c}, —N(R^{a})—CO₂R^{c}, —C(═O)N(R^{a}R^{b}), —SO₂N(R^{a}R^{b}), —N(R^{a})C(═O)R^{b}, —N(R^{a})CO₂R^{c}, —N(R^{a})SO₂R^{c}, —N(R^{a})SO₂N(R^{a}R^{b}), —OC(═O)N(R^{a}R^{b}), or —N(R^{a})C(═O)N(R^{a}R^{b}),
(4) —C₁₋₆ haloalkyl,
(5) —O—C₁₋₆ haloalkyl,
(6) —OH,
(7) halo,
(8) —NO₂,
(9) —CN,
(10) —C(═O)R^{a},
(11) —CO₂R^{c},
(12) —S(O)ₙR^{c},
(13) —SO₂N(R^{a}R^{b}),
(14) —N(R^{a}R^{b}),
(15) —C(═O)N(R^{a}R^{b}),
(16) 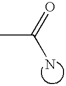
wherein 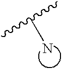
is azetidinyl, pyrrolidinyl, piperidinyl, or morpholino,
(17) —N(R^{a})SO₂R^{c},
(18) —OC(═O)N(R^{a}R^{b}),
(19) —N(R^{a})C(═O)N(R^{a}R^{b}),
(20) —N(R^{a})—C₁₋₆ alkyl-C(═O)N(R^{a}R^{b}),
(21) —N(R^{a})—C₁₋₆ alkyl-SR^{a},
(22) —N(R^{a})—C₁₋₆ alkyl-OR^{a},
(23) —N(R^{a})—C₁₋₆ alkyl-N(R^{a})₂,
(24) —N(R^{a})—C₁₋₆ alkyl-N(R^{a})—C(R^{a})═O,
(25) —N(R^{a})—C(═O)—C₁₋₆ alkyl-N(R^{a}R^{b}),
(26) —N(R^{a})C(═O)—C(═O)N(R^{a}R^{b}),
(27) —OCO₂R^{c},
(28) —N(R^{a})—SO₂N(R^{a}R^{b}),
(29) —N(R^{a})—SO₂—C₁₋₆ alkyl-N(R^{a}R^{b}),
(30) —N(R^{a})C(═O)R^{b},
(31) —N(R^{a})CO₂R^{c},
(32) —S—C₁₋₆ alkyl-C(═O)N(R^{a}R^{b}),
(33) —N(SO₂R^{c})—C₁₋₆ alkyl-C(═O)N(R^{a}R^{b}),
(34) —N(R^{a})—C(═O)—C₁₋₆ alkyl-C(═O)N(R^{a}R^{b}),
(35) —N(R^{a})—C(═O)—C₁₋₆ alkyl-N(R^{a})C(═O)(R^{b}),
(36) —N(R^{a})—SO₂—C₁₋₆ alkyl-C(═O)N(R^{a}R^{b}),
(37) —N(R^{a})—SO₂—C₁₋₆ alkyl-N(R^{a})C(═O)(R^{b}),
(38) —C(═O)N(R^{a})—C₁₋₆ alkyl-C(═O)N(R^{a}R^{b}),
(39) —C(═O)N(R^{a})—C₁₋₆ alkyl-N(R^{a})C(═O)(R^{b}), with the proviso that the —N(R^{a})— moieties are not both attached to the same carbon atom of the —C₁₋₆ alkyl- moiety,
(40) —C(═O)N(R^{a})—C₁₋₆ alkyl-O—C₁₋₃ alkyl, with the proviso that the —N(R^{a})— moiety and the —O—C₁₋₃ alkyl group are not both attached to the same carbon atom of the —C₁₋₆ alkyl- moiety, or
(41) —C(═O)N(R^{a})—C₁₋₆ alkyl-S(O)ₙR^{c};
Q¹ is:
(1) —H,
(2) —C(═O)N(R^{a}R^{b}),
(3) —C₁₋₆ alkyl-C(═O)N(R^{a}R^{b}),
(4) —S—C₁₋₆ alkyl-C(═O)N(R^{a}R^{b}),
(5) —O—C₁₋₆ alkyl-C(═O)N(R^{a}R^{b}),
(6) —N(R^{a})—C(R^{b})═O,
(7) —N(SO₂R^{c})—C₁₋₆ alkyl-C(═O)N(R^{a}R^{b}),
(8) —N(R^{a})—C(═O)—C(═O)—N(R^{a}R^{b}),
(9) —N(R^{a})SO₂R^{c},
(10) —SO₂N(R^{a}R^{b}),
(11) —CH═CH—C(═O)—N(R^{a}R^{b}),
(12) —N(R^{a})—C₁₋₆ alkyl-C(═O)N(R^{a}R^{b}),
(13) —N(R^{a})—C(═O)—N(R^{a}R^{b}),
(14) -HetC,
(15) —C₁₋₆ alkyl-HetC, or
(16) —N(R^{a})—C₁₋₆ alkyl-HetC;
HetC is a 5- to 7-membered saturated heterocyclic ring containing from 1 to 4 heteratoms independently selected from N, O and S, wherein the saturated heterocyclic ring is optionally substituted with from 1 to 4 substituents each of which is independently halogen, —C₁₋₄ alkyl, —C₃₋₆ cycloalkyl, —O—C₁₋₄ alkyl, —C₁₋₄ haloalkyl, —O—C₁₋₄ haloalkyl, —CN, oxo, phenyl, benzyl, phenylethyl, —(CH₂)₀₋₃C(═O)N(R^{a}R^{b}), —(CH₂)₀₋₃C(═O)R^{a}, —N(R^{a})—C(═O)R^{b}, N(R^{a})—CO₂R^{c}, —(CH₂)₁₋₃N(R^{a})—C(═O)R^{b}, —N(R^{a}R^{b}), —(CH₂)₁₋₃N(R^{a}R^{b}), —SO₂R^{c}, —(CH₂)₀₋₃C(═O)-HetD, -HetD, —N(R^{a})-HetD, and —(CH₂)₁₋₃-HetD; wherein each HetD is independently a 5- or 6-membered heteroaromatic ring containing from 1 to 4 nitrogen atoms or a 5- or 6-membered saturated heterocyclic ring containing from 1 to 4 nitrogen atoms, wherein the ring is optionally substituted with 1 or 2 substituents each of which is independently halogen, oxo, —C₁₋₄ alkyl, or —O—C₁₋₄ alkyl;
each R^{a} is independently —H, —C₁₋₆ alkyl, —C₁₋₆ haloalkyl, or —C₃₋₆ cycloalkyl;
each R^{b} is independently —H, —C₁₋₆ alkyl, —C₁₋₆ haloalkyl, or —C₃₋₆ cycloalkyl;
each R^{c} is independently —C₁₋₆ alkyl, —C₁₋₆ haloalkyl, or —C₃₋₆ cycloalkyl; and
each n is independently an integer equal to zero, 1, or 2.

2. The pharmaceutical composition according to claim 1, wherein the compound is a compound of Formula (II) or a pharmaceutically acceptable salt thereof: 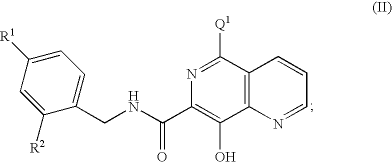
and a nonionic surfactant; wherein in Formula (II) each of R¹ and R² is independently:
(1) —H,
(2) —C₁₋₄ alkyl,
(3) —(CH₂)₁₋₃—C(═O)N(R^{a}R^{b}),
(4) —(CH₂)₁₋₃—N(R^{a}R^{b}),
(5) —(CH₂)₁₋₃—CO₂R^{c},
(6) —(CH₂)₁₋₃—S(O)ₙR^{c},
(7) —(CH₂)₁₋₃—SO₂N(R^{a}R^{b}),
(8) —O—C₁₋₄ alkyl,
(9) —CF₃,
(10) —OCF₃,
(11) halo selected from —F, —Cl, and —Br,
(12) —CO₂R^{c},
(13) —S(O)ₙR^{c},
(14) —SO₂N(R^{a}R^{b}),
(15) —N(R^{a}R^{b}), or
(16) —C(═O)N(R^{a}R^{b});
with the proviso that at least one of R¹ and R² is not —H;
Q¹ is —H, —C(═O)N(R^{a}R^{b}), —N(R^{a})SO₂R^{c}, or 1,1-dioxido-1,2-thiazinan-2-yl;
each R^{a} is independently —H, —C₁₋₄ alkyl, or cyclopropyl;
each R^{b} is independently —H, —C₁₋₄ alkyl, or cyclopropyl; and
each R^{c} is independently a —C₁₋₄ alkyl or cyclopropyl.

3. The pharmaceutical composition according to claim 2, wherein the compound of Formula (II) is Compound A or a sodium salt of Compound A, wherein Compound A is 5-(1,1-dioxido-1,2-thiazinan-2-yl)-N-(4-fluorobenzyl)-8-hydroxy-1,6-naphthyridine-7-carboxamide.

4. The pharmaceutical composition according to claim 1, wherein the nonionic surfactant is present in an amount of at least about 0.1 wt. %

5. The pharmaceutical composition according to claim 1, wherein the nonionic surfactant comprises a poloxamer.

6. The pharmaceutical composition according to claim 1, wherein the composition is encapsulated.

7. The pharmaceutical composition according to claim 6, wherein the encapsulated composition is a granulated composition further comprising a disintegrant, optionally a diluent, optionally a binder, optionally a lubricant, and optionally an antioxidant.

8. The pharmaceutical composition according to claim 6, wherein the compound in the encapsulated composition is Compound A or a sodium salt thereof; and the encapsulated composition further comprises a disintegrant.

9. The pharmaceutical composition according to claim 1, wherein the composition is compressed into a tablet.

10. The pharmaceutical composition according to claim 9, wherein the compressed tablet further comprises a diluent, a disintegrant, a lubricant, optionally a binder, and optionally an antioxidant.

11. The pharmaceutical composition according to claim 9, wherein the compound in the compressed tablet is Compound A or a sodium salt thereof; and the compressed tablet further comprises diluent A, a disintegrant, and a lubricant.

12. The pharmaceutical composition according to claim 9, wherein the compound in the compressed tablet is Compound A or a sodium salt thereof; and the compressed tablet further comprises a diluent, a disintegrant, a lubricant, and a stabilizing agent.

13. The encapsulated composition according to claim 7, wherein:
the compound is Compound A or a sodium salt thereof;
the nonionic surfactant comprises a poloxamer or a polysorbate;
the disintegrant comprises croscarmellose sodium, crospovidone, povidone, or sodium starch glycolate;
the optional diluent comprises lactose, microcrystalline cellulose, mannitol, anhydrous dibasic calcium phosphate, dibasic calcum phosphate dihyrate, or a combination of any two of the foregoing;
the optional binder comprises hydroxypropyl cellulose, hydroxypropyl methylcellulose, or povidone;
the optional lubricant comprises magnesium stearate, stearic acid, sodium stearyl fumarate, or a combination of any two of magnesium stearate, stearic acid, and sodium stearyl fumarate; and
the optional antioxidant comprises butylated hydroxyanisole.

14. The encapsulated composition according to claim 7, wherein the compound is Compound A or a sodium salt thereof; and the composition comprises from about 5 to about 80 wt. % Compound A sodium salt, from about 0.1 to about 20 wt. % nonionic surfactant, from about 0.5 to about 10 wt. % disintegrant, from 0 to about 90 wt. % diluent, from 0 to about 20 wt. % binder, from 0 to about 10 wt. % lubricant, and from 0 to about 0.1 wt. % antioxidant.

15. The compressed tablet composition according to claim 10, wherein:
the compound is Compound A or a sodium salt thereof;
the nonionic surfactant comprises a poloxamer or a polysorbate;
the diluent comprises lactose, microcrystalline cellulose, or both lactose and microcrystalline cellulose;
the disintegrant comprises croscarmellose sodium, crospovidone, povidone, or sodium starch glycolate;
the lubricant comprises magnesium stearate, stearic acid, sodium stearyl fumarate, or a combination of any two of magnesium stearate, stearic acid and sodium stearyl fumarate;
the optional binder comprises hydroxypropyl cellulose, hydroxypropyl methylcellulose, or povidone; and
the optional antioxidant comprises butylated hydroxyanisole.

16. The compressed tablet composition according to claim 10, wherein the compound is Compound A or a sodium salt thereof; and the compressed tablet comprises from about 5 to about 75 wt. % Compound A sodium salt, from about 0.1 to about 20 wt. % nonionic surfactant, from about 15 to about 90 wt. % diluent, from about 0.5 to about 10 wt. % disintegrant, from about 0.2 to about 10 wt. % lubricant, from 0 to about 10 wt. % binder, and from 0 to about 0.1 wt. % antioxidant.

17. A method for preparing an encapsulated pharmaceutical composition according to claim 8, wherein the method comprises:
(A) wet granulating a mixture of Compound A or Compound A sodium salt, the nonionic surfactant, and the disintegrant; and optionally then milling the wet granulated mixture;
(B) drying the wet granulated mixture of Step A;
(C) milling the dried mixture of Step B; and
(D) encapsulating the milled mixture of Step C.

18. The method according to claim 17, wherein the encapsulated composition further comprises a diluent, a lubricant, a binder, and an antioxidant; wherein the mixture employed in granulation Step A comprises Compound A or its sodium salt, the nonionic surfactant, the disintegrant, the diluent, the binder, and the antioxidant; and wherein the method further comprises lubricating the milled mixture from Step C with the lubricant prior to encapsulation in Step D.

19. The method according to claim 18, wherein Compound A is employed in the form of a sodium salt; the nonionic surfactant is poloxamer; the diluent is lactose and microcrystalline cellulose; the disintegrant is croscarmellose sodium; the binder is hydroxypropyl cellulose; the lubricant is sodium stearyl fumarate; and the antioxidant is BHA.

20. The method according to claim 19, wherein the encapsulated composition comprises from about 5 to about 40 wt. % Compound A sodium salt, from about 0.5 to about 15 wt. % poloxamer 407, from about 10 to about 40 wt. % lactose, from about 10 to about 40 wt. % microcrystalline cellulose, from about 0.5 to about 5 wt. % croscarmellose sodium, from about 0.2 to about 6 wt. % sodium stearyl fumarate, from about 0.1 to about 20 wt. % hydroxypropyl cellulose; and from about 0.01 to about 0.1 wt. % BHA.

21. A method for preparing a compressed tablet pharmaceutical composition according to claim 11, wherein the method comprises:
(A) wet granulating a mixture of Compound A or Compound A sodium salt, the nonionic surfactant, diluent A, the disintegrant; and optionally then milling the wet granulated mixture;
(B) drying the wet granulated mixture of Step A;
(C) milling the dried mixture of Step B; and
(D) lubricating the milled mixture of Step C with the lubricant; and
(E) compressing the lubricated mixture of Step D into a tablet.

22. The method according to claim 21, wherein the compressed tablet composition further comprises diluent B, optionally a binder, and optionally an antioxidant; and wherein the mixture employed in granulation Step A comprises Compound A or its sodium salt, the nonionic surfactant, the disintegrant, diluent A, diluent B, the binder (optional), and the antioxidant (optional).

23. The method according to claim 21, wherein the compressed tablet composition further comprises diluent B, optionally a binder, and optionally an antioxidant; wherein the mixture employed in granulation Step A comprises Compound A or its sodium salt, the nonionic surfactant, the disintegrant, diluent A, the binder (optional), and the antioxidant (optional); and wherein the method further comprises blending the milled mixture of Step C with diluent B prior to lubrication in Step D.

24. The method according to claim 21, wherein Compound A is employed in the form of a sodium salt; the nonionic surfactant is poloxamer; diluent A is lactose; the disintegrant is croscarmellose sodium; and the lubricant is magnesium stearate or sodium stearyl fumarate.

25. The method according to claim 22, wherein Compound A is employed in the form of a sodium salt; the nonionic surfactant is poloxamer; diluent A is lactose; the disintegrant is croscarmellose sodium; the lubricant is magnesium stearate or sodium stearyl fumarate; diluent B is microcrystalline cellulose; the binder is employed and is hydroxypropyl cellulose; and the optional antioxidant is BHA.

26. The method according to claim 23, wherein Compound A is employed in the form of a sodium salt; the nonionic surfactant is poloxamer; diluent A is lactose; the disintegrant is croscarmellose sodium; the lubricant is magnesium stearate or sodium stearyl fumarate; diluent B is microcrystalline cellulose; the binder is employed and is hydroxypropyl cellulose; and the optional antioxidant is BHA.

27. The method according to claim 24, wherein the compressed tablet comprises from about 10 to about 70 wt. % Compound A sodium salt, from about 0.5 to about 10 wt. % poloxamer, from about 10 to 50 wt. % lactose, from about 10 to about 50 wt. % microcrystalline cellulose, from about 0.5 to about 10 wt. % croscarmellose sodium, from about 0.2 to about 6 wt. % magnesium stearate or sodium stearyl fumarate, from about 0.5 to about 10 wt. % hydroxypropyl cellulose, and from 0 to about 0.1 wt. % BHA.

28. A method for preparing a compressed tablet pharmaceutical composition according to claim 12, wherein the method comprises:
(A) freeze-drying a suspension of Compound A or its sodium salt, the nonionic surfactant, and the stabilizing agent;
(B) dry mixing the freeze-dried product of Step A with the diluent, disintegrant, and lubricant; and
(C) compressing the mixture of Step B into a tablet.

29. A method for inhibiting HIV integrase in a subject in need of such inhibition, which comprises administering to the subject the pharmaceutical composition according to claim 1.

30. A method for preventing or treating HIV infection or for preventing, treating or delaying the onset of AIDS in a subject in need thereof, which comprises administering to the subject the pharmaceutical composition according to claim 1.
